(19) United States
(12) Patent Application Publication
(10) Pub. No.: US 2004/0219600 A1
(43) Pub. Date: Nov. 4, 2004

(54) Method for determining sensitivity to environmental toxins and susceptibility to parkinson's disease

(76) Inventors: Robert Wood Williams III, Memphis, TN (US); Richard Jay Smeyne, Collierville, TN (US); Michelle Smeyne, Collierville, TN (US); Ruby Cook Tharpe, Olive Branch, MS (US); Elizabeth Cook, Olive Branch, MS (US)

Correspondence Address: BUTLER, SNOW, O'MARA, STEVENS & CANNADA PLLC, 6075 POPLAR AVENUE, SUITE 500, MEMPHIS, TN, 38119, US

(21) Appl. No.: 10/734,372

(22) Filed: Dec. 12, 2003

Related U.S. Application Data
(60) Provisional application No. 60/433,437, filed on Dec. 13, 2002.

(51) Int. Cl. G01N 33/53; G01N 33/567; C12Q 1/48

(52) U.S. Cl. 435/7.1; 435/15

(57) ABSTRACT

The present invention relates to the identification of a gene encoding the protein glutathione S-transferase pi2 as being correlated with the susceptibility to neurotoxicity and concomitantly the risk to develop Parkinson's disease. Based on this correlation, various methods are disclosed, including determining the amount of the one or more glutathione S-transferase present in a biological sample in response to an environmental toxin, to assess the risk to develop Parkinson's disease.

This application is a continuation-in-part of Provisional Application Ser. No. 60/433,437 filed on Dec. 13, 2002, (hereby incorporated by reference in its entirety). Applicants' claim the benefit of this application under 35 U.S.C. § 119(e).

This invention was made with government support under grants 42077 and NS39006 awarded by the National Institutes of Health. The government has certain rights in the invention.

### BACKGROUND OF THE INVENTION

1. Field of the Invention

The present invention relates to the role of glutathione S-transferase in Parkinson's disease and in sensitivity of an individual to environmental toxins. It relates to a method of assessing the susceptibility of an individual to environmental toxins and to Parkinson's disease.

2. Description of the Related Art

The syndrome, which we know today as Parkinson's disease, was first described in 1817 by Dr. James Parkinson in a paper entitled “An Essay on the Shaking Palsy” (Parkinson, J., An Essay on Shaking Palsy, Sherwood, Neeley and Jones, London, 1817). Parkinson's disease is a debilitating neurological disorder that strikes approximately 1-2% of the adult population greater than 50 years of age (new incidence is 20 per 100,000 persons) (de Rijk M C, et al., Neuro. 45:2143-46 (1995)). Current estimates from the American Parkinson's Disease Foundation put the number of American citizens suffering from this disease at greater than 1,000,000 persons. The costs of treatment of Parkinson's disease can be staggering. At an average per patient cost of $6000.00 per year (includes drugs, physicians and loss of pay to patient and family members) (Whetten-Goldstein K, et al., The Burden of Parkinson's Disease On Society, Family, And The Individual, J. Am. Ger. Soc. 45:479-85 (1997)), the total cost of the disease may approach $6,000,000,000.00 per year; of which 85% is borne to private and government (Social Security, Medicare) insurance. In fact, more individuals present with Parkinson's disease than with multiple sclerosis, muscular dystrophy and amyotrophic lateral sclerosis (Lou Gehrig's Disease) combined (The Parkinson's Web, 1997). Since the population of the world is, on average, getting progressively older (United States Census Bureau, 1996), the number of people suffering from this disease should substantially increase within the next several decades. Since Parkinson's disease is an incurable disease with an average life expectancy after diagnosis of 15 years, there should be an even larger burden on both the social and financial resources of families, insurance companies and the Federal government than is present today.

Parkinson's disease is characterized by a loss of the pigmented cells located in the midbrain substantia nigra pars compacta (SNpc). The loss of these cells results in a reduction in the number of the afferent fibers that project to the striatum. Parkinson's disease symptoms first manifest when approximately 60% of the SNpc neurons have already died (German, D. C., et al., Midbrain Dopaminergic Cell Loss In Parkinson's Disease: Computer Visualization, Annals of Neuro., 26:507-14 (1989)). Because the progression of cell loss is thought to occur over a somewhat protracted period of time in a defined spatiotemporal manner (Damier, P., et al., The Substantia Nigra of the Human Brain. II. Patterns of Loss of Dopamine-Containing Neurons in Parkinson's Disease Brain, 122:1437-48 (1999)), the onset of Parkinson's disease symptoms are often insidious.

The underlying cause for the vast majority of Parkinson's disease cases is unknown. Controversy still exists as to how much of the disease results from a strict genetic causation, a purely environmental factor, or the more parsimonious combination of the two risk factors (Duvoisin, R. C., Genetic and Environmental Factors in Parkinson's Disease, Adv. Neuro., 80: 161-3 (1999)). Empirical evidence suggests that less than 10% of all diagnosed Parkinsonism has a strict familial etiology (Payami, H. and Zareparsi, S., Genetic Epidemiology of Parkinson's Disease. [Review] [133 refs], J. of Geriatric Psych. & Neuro. 98-106 (1998)). A small number of familial parkinsonian patients appear to have a polymorphism in the alpha-synuclein gene (Polymeropoulos, M. H., et al., Mutation in the Alpha-Synuclein Gene Identified in Families with Parkinson's Disease., Science, 276:2045-047 (1997)), suggesting that this aggregating protein may play a role in Lewy body formation that ultimately results in substantia nigra cell death (Nussbaum, R. L., et al., M.H., Genetics of Parkinson's Disease, Hum. Mol. Gen., 6:1687-92 (1997)). A second autosomal recessive locus coding for the Parkin protein maps to the long arm of chromosome 6 (6q25.2-q27). Mutations in this gene have been shown to cause a form of juvenile onset Parkinson's disease. Other genes that have been shown to have an association with Parkinson's disease include loci at human chromosome 2p 13 and 4p (Gasser T, Genetics of Parkinson's Disease, J. Neuro. 248:833-40 (2001)). The Parkinson's disease linked to this locus more closely resembles that of idiopathic Parkinson's disease, although like the alpha-synuclein protein, this unknown protein has very low penetrance. However, at this time no mutations in these proteins have been reported in idiopathic Parkinson's disease.

Since the vast majority of Parkinson's disease patients have no direct tie to any identified genetic mutation, important information regarding the pathophysiology of Parkinson's disease may be gleaned through the study of animal models. The discovery of the neurotoxin 1-methyl-4-phenyl-1,2,3,6-tetrahydropyridine (MPTP) has provided a useful model of Parkinsonism that appears to recapitulates the pathology of the disease seen in man. MPTP was first described in studies of heroin addicts who presented with symptoms indistinguishable from those of Parkinson's disease (Burns, R. S., et al., The Clinical Syndrome of Striatal Dopamine Deficiency; Parkinsonism Induced by MPTP, N. Engl. J. Med., 312:1418-21 (1985)). MPTP has been demonstrated to exert its neurotoxic effects in humans and other primates, cats, and in some rodents. In rodents, it has been shown that only specific strains of mice are sensitive to the administration of MPTP (Sundstrom, E., et al., Studies on the Effect of 1-methyl-4-phenyl-1,2,3,6-tetrahydropyridine (MPTP) on Central Catacholamine Neurons in C57B1/6 Mice Comparison with Three Other Strains of Mice, Brain Res., 405:26-38 (1987)).

Parkinson's disease, once diagnosed, is progressive. As stated earlier, one of the major problems in the treatment of Parkinson's disease is that a majority of the cells of the SNpc (approximately 60%) are lost prior to the onset of symptoms (Brucke T, et al., Measurement of the Dopaminergic Degeneration in Parkinson's Disease with [1231] beta-CIT and SPECT. Correlation with Clinical Findings and Comparison with Multiple System Atrophy and Progressive Supranuclear Palsy, J. of Neural. Trans. Suppl. 50:9-24 (1997)). Therefore, if one could identify individuals at higher risk for developing Parkinson's disease, or identify individuals at an earlier time, then both pharmacological and nonpharmacological treatments could be instituted which might slow the cell loss such that patients can defer or even stop SNpc cell loss from reaching the critical 60% level. If this happens, the disease process may not be cured, but, functionally, the disease symptoms would be eliminated.

The underlying cause of idiopathic Parkinson's disease is unknown. Although several genes, such as alpha-synuclein and Parkin have been linked to an early-onset form of substantia nigra degeneration similar to that seen in Parkinson's disease (Gasser, T., Genetics of Parkinson's Disease, J. Neuro., 248:833-40 (2001)), no mutations in these genes has been reported in the typical adult-onset type of Parkinson's disease (Scott, W. K., et al. The alpha-synuclein gene is not a major risk factor in familial Parkinson disease, Neurogenetics, 2:191-192 (1999)). Current hypothesis posit that idopathic Parkinson's disease results from a genetic susceptibility to an environmental agent (Duvoisin, R. C., Genetic and Environmental Factors in Parkinson's Disease, Adv. Neuro., 80:161-3 (1999)). MPTP is a neurotoxin whose administration causes a variable Parkinsonian syndrome that is similar to that seen in idopathic Parkinson's disease (Snow, B. J., et al., Pattern of Dopaminergic Loss in the Striatum of Humans with MPTP Induced Parkinsonism, J. of Neuro., Neurosurgery & Psychiatry, 68:313-16 (2000)). Structurally, MPTP is similar to many common pesticides and herbicides (Nagatsu, T. et al., Inhibition of the Tyrosine Hydroxylase System by MPTP, 1-methyl-4-phenylpyridinium ion (MPP+) and the Structurally Related Compounds in Vitro and in Vivo, Euro. Neuro., 26 Suppl. 1 11-5 (1987)) which may be the reason that idopathic Parkinson's disease has a higher incidence in agricultural communities (Gorell, J. M., et al., The Risk of Parkinson's Disease with Exposure to Pesticides, Farming, Well Water, and Rural Living, Neuro., 50:1346-50 (1998)). It is not known, however, if the cellular mechanisms, and therefore the genetic susceptibilities of each of these compounds are similar or whether each affects substantia nigra degeneration through different pathways. This could be tested by determining quantitative trait loci for other neurodegenerative compounds as they relate to SNpc neuron degeneration.

There have been a few studies that examined the expression of the various classes of glutathione S-transferase enzymes in the brain (Johnson, T. E., et al., Mapping Quantitative Trait Loci for Behavioral Traits in the Mouse, Behav. Genet., 22:635-53 (1992)), although none have examined their expression in the anatomical circuits underlying Parkinson's disease. Similarly, no identified genes have been directly correlated with risk for development of idiopathic Parkinson's disease.

### BRIEF SUMMARY OF THE INVENTION

This invention relates a method to determine the risk of developing Parkinson's disease in an individual. The method of this embodiment involves determining the expression levels of one or more glutathione S-transferases, that being by determining the amount of mRNAs to one or more glutathione S-transferases in a biological sample and comparing this level of expression of one or more glutathione S-transferases levels with the level of expression of one or more glutathione S-transferases after exposure of the biological sample to an environmental toxin.

One embodiment of a genetic screen for Parkinson's disease involves determining the level of susceptibility of a subject to an environmental toxin capable of detoxification by one or more glutathione S-transferases. This method involves determining a first amount of one or more glutathione S-transferases present in a biological sample from the subject, contacting the biological sample with the environmental toxin, and determining a second amount of one or more glutathione S-transferases present in the biological sample after the biological sample has been contacted with the environmental toxin. If the second amount of one or more glutathione S-transferases is lower than or similar to the first amount of one or more glutathione S-transferases this indicates that the subject has a higher level of susceptibility than a subject having the second amount of one or more glutathione S-transferases higher than the first amount. Similarly, if the second amount of one or more glutathione S-transferases is higher than the first amount of one or more glutathione S-transferases this indicates the subject has a lower level of susceptibility than a subject having a second amount of one or more glutathione S-transferases lower than or similar to the first amount. If a subject has a higher level of susceptibility to an environmental toxin then the subject has a higher risk of developing Parkinson's disease.

This invention identifies a gene, encoding the protein glutathione S-transferase pi2 (GSTpi2), as being correlated with the risk for development of idiopathic Parkinson's disease. This invention also relates to an isolated cDNA sequence for glutathione S-transferase pi2. The DNA sequence expressing the amino acid sequence encoding the glutathione S-transferase pi2 protein is set out in SEQ ID NO. 1. The identification of this genetic correlation allows for the development of genetic screens for the sensitivity to environmental toxins and the risk of developing Parkinson's disease.

More specifically, this invention provides a method to determine genetic susceptibility to an environmental toxin involving contacting a biological sample from a subject with one or more labeled probes each of which binds selectively to a target sequence being a region on mouse chromosome 1, spanning a 20 cM region from D1Mit113 to D1Mit293 under conditions in which the one or more labeled probes form a stable hybridization complex with the target sequence and detecting the hybridization complex. The presence of the hybridization complex being indicative of a genetic susceptibility to an environmental toxin. This region is homologous (81% identity from mouse to human) to a region of human chromosome 11, in a region spanning nucleotides 69874216-69877057.

This invention also provides a method to determine the susceptibility of a subject to develop Parkinson's disease. The method involves contacting a biological sample from a subject with a composition consisting essentially of a plurality of labeled probes each of which selectively binds to a region of human Glutathione S-transferase pi within a segment of human GSTP1 (chr11:69874218-69877056); GST mu (hGSTm4: chr1:110677671-110687021, hGSTm2: chr1:110689771-110696957, hGSTm3: chr1:110758175-110761973, hGSTm1:chr1:110709530-110715415; or GST alpha (hGSTa1:chr6:52658645-52670705, hGSTa2:chr6: 52617245-52630341; hGSTa3:chr6: 52763514-52776547, hGSTa4:chr6:52844814-52862163 under conditions which a plurality of labeled probes form stable hybridization complexes with a segment of human GSTP1 (chr11:69874218-69877056); GST mu (hGSTm4: chr1:110677671-110687021, hGSTm2: chr1:110689771-110696957, hGSTm3: chr1:110758175-110761973, hGSTm1:chr1:110709530-110715415; or GST alpha (hGSTa1:chr6:52658645-52670705, hGSTa2:chr6:52617245-52630341; hGSTa3:chr6: 52763514-52776547, hGSTa4:chr6: 52844814-52862163; and detecting the hybridization complex wherein said hybridization complex is indicative of increased susceptibility of a subject to develop Parkinson's disease.

### DETAILED DESCRIPTION OF THE INVENTION

The present invention may be understood more readily by reference to the following detailed description of specific embodiments and the Examples included herein.

The present invention provides the identification of a candidate gene for MPTP-sensitivity, and, concomitantly the risk of developing Parkinson's disease. With this correlation established, genetic screens for the susceptibility to develop Parkinson's disease can be developed for various glutathime S-transferases. Idiopathic Parkinson's disease, like many diseases and biological phenotypes, is thought to arise as a consequence of the interplay of two or more genes (Duvoisin, et al. 1999, Genome-Wide Scan for Parkinson's Disease: The Gene Parkinson's Disease Study, Neuro., 57:1124-26 (2001)). The cumulative phenotypes that arise from the interaction of these differing gene effects are termed quantitative traits. In fact, differential responses to various exogenous substances such as environmental toxins are thought to be influenced by the interactions of multiple gene products (McClearn, G. E. et al., The Utilization of Quantitative Trait Loci in Toxicogenetics., J. Exp. Anim. Sci., 35:251-58 (1993)). One method that has been used to identify multiple genes that present an additive phenotype is termed quantitative trait loci (QTL) analysis. The premise behind QTL analysis is that if numerous genetic markers are examined, only those that segregate with a particular phenotype will contain the gene(s) that underlie the trait being examined (Crusio, W. E., Quantitative Genetics. In D. Goldowitz, et al. (Ed.), Techniques for the Genetic Analysis of Brain and Behavior, Elsevier, Amsterdam, pp. 231-50 (1992)). QTL analysis in mice has been facilitated in the last ten years by several significant advances. First, thousands of microsatellite markers have been found and mapped to their specific chromosomal region spanning the entire murine genome (Brown, S. D., The Mouse Genome Project and Human Genetics. A Report From the 5th International Mouse Genome Mapping Workshop, Lunteren, Holland., Genomics, 13:490-92 (1992)). Second, several strains of mice have been found that respond differently to a variety of external stimuli (Gerlai, R., Gene-targeting Genetics of Parkinson's Disease, J. Neuro., 248:833-40 (2001)). Third, the raw sequence of the mouse genome has been virtually completed allowing for substantial bioinformatic work to be done on gene identification.

We, and others (Heikkila, R. E., Differential Neurotoxicity of 1-methyl-4-phenyl-1,2,3,6-tetrahydropyridine (MPTP) in Swiss-Webster Mice from Different Sources, Eur. J. Pharmacol., 117:31-133 (1985)), have found that there is a differential sensitivity to MPTP among various strains of mice. In the present invention, we have used quantitative trait loci analysis to examine differences in MPTP sensitivity between inbred C57B1/6J and Swiss Webster mice as well as in individual hybrid strains derived from crosses of these founder lines. Using information on the chromosomal polymorphisms evident between these two strains, we have identified the chromosomal region that is responsible for differences between the strains in regard to the MPTP sensitivity. Specifically, the present invention provides glutathione S-transferase pi2 as a candidate gene located within our identified chromosomal region.

Glutathione S-transferases (GSTs) are a class of very abundant proteins in eukaryotes that appear to function as xenobiotic metabolizing proteins (Ketterer B, et al., Enzymology of Cytosolic Glutathione S-transferases, Adv. Pharmacol. 27:37-69 (1994)). This means that this class of enzymes can be viewed as a cell defense against numerous manmade and naturally-occurring environmental agents. Structurally, the active glutathione S-transferase is composed of dimers containing one of four subunit classes: alpha, mu, omega, or pi (Salinas A E, et al., Glutathione S-transferases—A Review, Curr. Med. Chem. 6:279-09 (1999)). Zhan-Li Yan, et al., Immune Histochemistry of Omega Class Glutathione S-Transferase in Human Tissues; 49(8), J. Histochem. Cytochem. 983 (2001). Although each monomer of glutathione S-transferase contains essential residues, the quaternary structure of the holoenzyme is critical since each monomer contributes half of an overall active site (Dirr H, et al., X-Ray Crystal Structures of Cytosolic Glutathione S-transferases. Implications for Protein Architecture, Substrate Recognition and Catalytic Function, Eur. J. Biochem. 220:645-61 (1994)). Functionally, glutathione S-transferases work by adding glutathione to electrophiles within any number of chemical structures (Hayes J D, et al., Glutathione S-transferase Polymorphisms and Their Biological Consequences, Pharma. 61:154-66 (2000)), including numerous by-products of oxidative stress. Each member (alpha, mu, omega, and pi) of the glutathione S-transferase family appears to function in a similar manner. Therefore, the cell-specific function of each glutathione S-transferase member will be dependent on its temporal and spatial expression.

Based upon the Ensemble and USC mouse genome server databases, genes encoding the various classes of glutathione S-transferases have been localized on several different chromosomes. To date, a total of three alpha class glutathione S-transferases, six mu class glutathione S-transferases and three pi class glutathione S-transferases have been found. For the alpha class; two genes are found in tandem on chromosome 1 (GSTa3 at chr1:21444565-21469162 and GSTa2 at Chr1:21489318-21520180) and one gene has been found on chromosome 9 (Gsta4 at chr9:78924864-78942169). For the mu class glutathione S-transferases, four genes have been found in tandem on chromosome 3 (Gstm6 at chr3:108432582-108436989; Gstm5 at chr3:108389178-108392010; Gstm2 at chr3:108475375-108480074; Gstm1 at chr3:108504693-108510414), while one gene is situated on chromosome 1:142165074-142165664). For the pi class, two genes (GSTp1a and 1b) are found in tandem on chromosome 19 (Gstp1a/b at chr 19:6764325-6766087), while one (glutathione S-transferase pi2) is found on chromosome 1 (GSTpi2 at Chr1: 193607013-193607642). Isozyme subunits within the same gene class are highly homologous (>75%), while homology between classes is in the range of 25-45%.

The rapid progress of genome sequencing has lead to a revitalized interest in the search for disease genes. In the past, several methods have been used to identify genes linked to disease, including segregation analysis and positional cloning. These types of analyses have been most successful when single genes are structurally mutated. When diseases occur in response to changes in interactions of multiple genes or through differences in gene expression, these types of analyses become more difficult.

We used quantitative trait loci analysis to identify regions of the genome that underlie the previously observed strain differences seen in mice following administration of MPTP. Quantitative trait loci (QTL) identify a gene or set of genes that have variant forms—or alleles—such that their differential expression or sequence produces measurable quantitative effects (Lynch, M. and Walsh, B., Genetics and Analysis of Quantitative Traits, Sinauer, Sunderland M A., (1998)). QTLs have been used to measure many types of biological variation in plants (Ming, R., et al., QTL Analysis in a Complex Autopolyploid: Genetic Control of Sugar Content in Sugarcane, Genome Res., 11:2075-84 (2001)) and animals. In humans, QTL analysis is often referred to as detection of susceptibility genes and has been used to identify behavioral traits (Bennett, B., Congenic Strains Developed for Alcohol- and Drug-Related Phenotypes, Pharmacol, Biochem. Behav., 67:671-81 (2000)), differences in learning abilities as well as factors that affect complex cellular processes leading to the development of neoplasms.

QTLs that have particularly large effects that verge on producing Mendelian segregation patterns are major-factor or major-effect QTLs. The large effects make them the easiest QTLs to map. Previously, we have shown that the sensitivity seen in C57BL/6 mice to MPTP is effectively a Mendelian dominant trait (Hamre, K., et al., Differential Strain Susceptibility Following 1-methyl-4-phenyl-1,2,3,6-tetrahydropyridine (MPTP) Administration Acts in an Autosomal Dominant Fashion: Quantitative Analysis in Seven Strains of Mus Musculus, Brain Res., 828: 91-103 (1999)), suggesting that one locus may underlie the majority of effects. Our results support this finding by demonstrating that a single major effect QTL located on chromosome 1 can account for much of the observed strain differences. However, we should be quick to add that the anticipated effect of the Mptp1 locus runs against expectation, and the C57BL/6J allele has protective effects. This strongly implicates the existence of a set of two or more epistatic loci that interact with the Chromosome 1 locus on the parental strain to convert a resistant allele into a sensitizing allele.

The power of a QTL analysis, especially in identifying human disease prevalence genes, is predicated on the identification of an appropriate phenotypic marker that is variable, is under partial genetic control, and that can be accurately phenotyped. In the present invention, we were interested in identifying a gene(s) whose expression was correlated with loss of dopamine functioning following administration of the parkinsonian toxin MPTP. To measure dopamine loss, we directly counted the number of SNpc cells seven days following SC administration of MPTP. While determination of cell number can be time consuming, we felt it was a more stable and accurate measure of the effect of this toxin since other indirect measures of MPTP's effects such as dopamine content in the striatum or levels of pre-synaptic striatal dopamine transporters (DATs) or dopamine D(2) receptors (D2R) have been shown to be plastic. Additionally, other indirect measures of damage to the SNpc such as behavioral changes in motor activity have also been shown to recover over time (Brooks, A. I., et al., Paraquat Elicited Neurobehavioral Syndrome Caused by Dopaminergic Neuron Loss, Brain Res., 823:1-10 (1999)).

Examination of identified genes located within the chromosome 1 QTL (Table 1) using the mouse genome database (http://www.informatics.jax) shows that only one has a known function that could be part of the toxification or detoxification mechanism of MPTP. This gene is called Gstp-rs1. It is also known as glutathione S-transferase pi2 (Hatayama et al., A cDNA Sequence Coding a Class Pi Glutathione S-Transferase of Mouse. Nuc. Acids Res. 18:1406 1990)).

TABLE 1
List of identified genes on mouse chromosme 1
within QTL scan width

| | |
|---|---|
| 96.0 | Fmn2, formin2 |
| 97.0 | Exol, exomiclease 1 |
| 97.3 | ic, ichthyosis |
| 97.6 | D1Ertd704 e, DNA segment, Chr 1, ERATO Doi 704, expressed |
| 97.6 | Hkpl, house-keeping protein 1 |
| 98.0 | Scfrl, stem cell frequency regulator 1 |
| 98.0 | Tlr5, toll-like receptor 5 |
| 98.5 | Ephxl, epoxide hydrolase 1, microsomal |
| 98. | Adprtl, ADP-ribosyltransferase (NAD+; poly (ADP-ribose) |
| | polymerase) |
| 98.7 | D1Ertd10e, DNA segment, Chr 1, ERATO Doi 10, expressed |
| 98.7 | D1Ertd578e, DNA segment, Chr 1, ERATO Doi 578 expressed |
| 98.7 | D1Ertd86e, DNA segment, Chr 1, ERATO Doi 86, expressed |
| 98.7 | Enah, enabled homolog (Drosophila) |
| 99.3 | Rgs7, regulator of G protein signaling 7 |
| 99.5 | Hlx, H2O-like homeo box gene |
| 99.7 | Chml, choroideremia-like |
| 100.0 | H25, histocompatibility 25 |
| 100.0 | Itpkb, inositol 1,4,5-trisphosphate 3-kinase B |
| 100.6 | Akpl, alkaline phosphatase 1 |
| 101.0 | D1Ertd75e, DNA segment, Chr 1, ERATO Doi 75, expressed |
| 101.5 | Tgfh2, transforming growth factor beta 2 |
| 102.0 | Gstp-rsl, glutathione S-trans ferase, pi, related sequence 1 |
| 103.0 | Nek2, NIMA(never in mitosis gene a)-related expressed kinase 2 |
| 103.4 | D1Ertd396e, DNA segment, Chr 1, ERATO Doi 396, expressed |
| 104.0 | D1Ertd654e, DNA segment Chr 1, ERATO Doi 654, expressed |
| 104.0 | Lamb3, laminin beta 3 |
| 105.0 | Traf5, Tnf receptor-associated factor 5 |
| 106.0 | Slc30al, solute carrier family 30 (zinc transporter), member 1 |

Glutathione S-transferase pi2 is encoded by a single exon containing 630 base pair. The predicted protein has 210 amino acid residues and a molecular weight of 23626.06 Daltons. The nucleotide sequence is highly similar to the GSTpi genes located on chromosome 19, although there is significant variation in the flanking noncoding regions. These flanking regions can be used to generate specific probes to differentiate the glutathione S-transferase family members for in situ and real-time PCR analysis of mRNA expression.

Using real time PCR technology we are able to measure relative or absolute mRNA levels in from very small sample pools. The RT-PCR reaction includes a forward primer, a primer with a fluorescent 5′ reporter dye and a 3′ quencher dye, and a reverse primer, constructed to hybridize with the target of interest. The 5′ to 3′ activity of the DNA polymerase cleaves the primer between the reporter and the quencher only if the primer hybridizes to the target. The primer is then displaced from the target, polymerization of the strand continues, and the produce accumulates with each cycle. The exponential amplification of product correlates with the initial number of target molecules when normalized to an endogenous reference, and the threshold cycle calculated (a cycle during which there is an exponential accumulation of produce occurring), as long as the efficiency of the target amplification and the efficiency of the reference amplification are approximately equal. To examine the mRNA expression levels of GSTp2, we used real time PCR analysis to examine baseline levels in C57B1/6 and SW mice. Preliminary results show that baseline levels GSTp2 from substantia nigra of C57B1/6 are higher than in seen in Swiss-Webster mice. Eight hours after the first injection of MPTP, expression of GSTp2 increases in C57B1/6 mice, but this level falls precipitously to 50% expression at 24 hours. The expression pattern in SW mice is different. Here, GSTp2 levels decrease 8 hours after administration of MPTP, but return to baseline levels at 24 hours.

Examination of mouse: human genetic homology from the Ensemble genome servers shows that there is a highly homologous hGSTpi on human chromosome 11. The mouse glutathione S-transferase pi2, located on mouse chromosome 1, has a genomic sequence (SEQ ID NO. 1) as follows:

ATGCCGCCGTACACCATTGTCTACTTCCCAAGTCGAGGGCGGTGTGAGGC
CATGCGCATGCTGCTGACTGACCAGGGCCAGAGCTGGAAGGAGGAGGTGG
TTACCATAGATACCTGGATGCAAGGCTTGCTCAAGCCCACTTGTCTGTAT
GGGCAGCTTCCTAAGTTTGAGGATGGAGACCTCACCCTTTACCAATCTAA
TGCCATCTTGAGACACCTTGGCCGCTCTTTGGGGCTTTATGGGAAAAACC
AGAGGGAGGCCGCCCAGGTGGATATGGTGAATGATGGGGTAGAGGACCTT
CGCGGCAAATATGGCACCATGATCTACAGAAACTATGAGAATGGTAAGAA
TGACTACGTGAAGGCCCTGCCTGGGCATCTGAAGCCTTTTGAGACCCTGC
TGTCCCAGAACCAGGGAGGCAAAGCTTTCATCGTGGGTGACCAGATCTCC
TTTGCCGATTACAACTTGCTGGACCTGCTGCTGATCCACCAAGTCCTGGC
CCCTGGCTGCCTGGACAACTTCCCCCTGCTCTCTGCCTATGTGGCTCGCC
TCAGTGCCCGGCCCAAGATCAAGGCCTTTCTGTCCTCCCCGGAACATGTG
AACCGTCCCATCAATGGCAATGGCAAACAG

The human GSTpi is 87% identical to the mouse glutathione S-transferase pi2 (Table 2), wherein human GSTpi is shown as SEQ ID NO. 3. The underlined portion of SEQ ID NO. 1 being set out as SEQ ID NO. 2 in Table 2.

TABLE 2

Mouse: human homology for glutathione S-transferase pi2.
There is an 87.8% identity from mouse to human.

| | | |
|---|---|---|
| M00000048 | ggccatgcgcatgctgctgactgaccagggccagagctggaaggaggaggtggt | M00000101 |
| | \|\|\|\| \|\|\|\|\|\|\|\|\|\|\|\|\|\| \| \|\| \|\|\|\|\|\|\|\|\|\|\|\|\|\|\|\|\|\|\|\|\|\|\|\|\|\|\|\|\| | |
| H69874878 | ggccctgcgcatgctgctggcagatcagggccagagctggaaggaggaggtggt | H69874931 |
| M00000151 | gggcagcttcctaagtttgaggatggagacctcaccctttacca | M00000194 |
| | \|\|\|\|\|\|\|\| \|\| \|\|\|\|\| \|\|\|\| \|\|\|\|\|\|\|\|\|\|\|\|\|\| \|\|\|\|\| | |
| H69875095 | gggcagctccccaagttccaggacggagacctcaccctgtacca | H69875138 |
| M00000350 | atgactacgtgaaggccctgcctgggcatctgaagccttttgagaccctg | M00000399 |
| | \|\|\|\|\|\|\| \|\|\|\|\|\|\|\| \|\|\|\|\| \|\|\|\|\| \|\|\|\|\|\|\|\|\|\|\|\|\|\|\|\|\|\|\|\|\| | |
| H69876521 | atgactatgtgaaggcactgcccgggcaactgaagccttttgagaccctg | H69876570 |
| M00000400 | ctgtcccagaaccagggaggcaa | M00000422 |
| | \|\|\|\|\|\|\|\|\|\|\|\|\|\|\|\|\|\|\|\|\|\|\| | |
| H69876571 | ctgtcccagaaccagggaggcaa | H69876593 |
| M00000442 | cagatctcctttgccgattacaacttgctggacctgctgctgatccacca | M00000491 |
| | \|\|\|\|\|\|\|\|\|\|\| \|\| \|\| \|\|\|\|\|\| \|\|\|\|\|\|\|\| \|\|\|\|\|\|\|\|\|\|\|\|\| \| | |
| H69876790 | cagatctccttcgctgactacaacctgctggacttgctgctgatccatga | H69876839 |
| M00000492 | agtcctggcccctggctgcctggacaacttccccctgctctctgcctatg | M00000541 |
| | \|\|\|\|\| \|\|\|\|\|\|\|\|\|\|\|\|\|\|\|\|\| \|\|\|\|\|\|\|\|\|\|\|\|\|\| \|\| \|\|\|\| | |
| H69876840 | ggtcctagcccctggctgcctggatgcgttccccctgctctcagcatatg | H69876889 |
| M00000542 | tggctcgcctcagtgcccggcccaagatcaaggcctttctgtcctccccg | M00000591 |
| | \|\|\| \|\|\|\|\|\|\|\|\|\|\|\|\|\|\|\|\|\|\|\|\| \|\|\|\|\|\|\|\|\|\| \|\|\| \|\|\|\|\|\|\| | |
| H69876890 | tggggcgcctcagtgcccggcccaagctcaaggccttcctggcctcccct | H69876939 |

| | | | |
|---|---|---|---|
| M00000592 | gaacatgtgaaccgtcccatcaatggcaa | (SEQ ID NO. 2) | M00000620 |
| | \|\| \| \|\|\|\|\|\|\| \|\|\|\|\|\|\|\|\|\|\|\|\|\| | | |
| H69876940 | gagtacgtgaacctccccatcaatggcaa | (SEQ ID NO. 3) | H69876968 |

In addition to identified genes, numerous unknown est sequences are also present in this region of chromosome 1. Using the bioinformatics packages inside the Celera database we can identify putative exon sequences. We have examined a list of putative exon sequences in this region by BLAST analysis to see if any of these putative exons are related to any known genes that may function in a neuroprotective/neurodegenerative pathway. At this time, we have found no genes, other than glutathione S-transferase pi2 which fits this criteria.

The nucleic acid sequence encoding glutathione S-transferase pi2 may be extended using a partial nucleotide sequence and employing various methods known in the art to detect upstream sequences such as promotes and regulatory elements. For example, one method which may be used is restriction-site PCR. This method uses the universe primers to retrieve unknown sequences adjacent to known locus (Sarker, G, PCR Meth. Appl. 2:318-22 (1993)). For example, genomic DNA is first amplified in the presence of a primer to a linker sequence and a primer specific to the known region. The amplified sequences then subject to a second round of PCR with the same linker primer and another specific primer internal to the first one. Products of each round PCR are transcribed with an appropriate RNA polymerase and sequences using reverse transcriptase.

Another method which may be used to retrieve unknown sequences is that of (Parker, J. D. et al., Nuc. Acids. Res. 19:3055-60 (1991)). Additionally, one may use PCR, nested primers, and various mouse databases such as Ensemble and Ceflera to identify the genomic DNA. Based on several databases, the GSTp2 located in mChr1 is encoded by a single exon. A similar gene on mChr19, which encodes a highly similar protein (>95% similar) is coded for by 5 exons. At this time, the promoter for either gene has yet to be identified, although it is assumed to lie in a region not greater that 10 mb 5′ of the initiation sequence of each gene.

The cDNA sequence of glutathione S-transferase pi2 can be cloned into any suitable expression vector, such as, for example, plasmid DNA, viral DNA including human viruses and invertebrate viruses and bacteriophages to form a recombinant expression system which directs the expression of glutathione S-transferase pi2 of the invention. It is understood that this expression system can be expressed in any suitable host cell to form a functional recombinant glutathione S-transferase pi2 protein.

The identification of a gene encoding GSP pi2 in mice and its homolog, glutathione S-transferase pi in humans, as being correlated with the risk for the development of idiopathic Parkinson's disease (as shown in Example 1), allows for the development of genetic screens for the sensitivity to environmental toxins and the risk of the development of Parkinson's disease. More specifically, the susceptibility of a subject to an environmental toxin capable of detoxification by glutathione S-transferase and the concomitant, risk of developing Parkinson's disease, can be assessed by various methods. In the first step of one of these methods, a first amount of one or more glutathione S-transferases present in a biological sample are determined. The term “one or more glutathione S-transferases” means any of isoenzymes of glutathione S-transferase, such as glutathione S-transferase pi2 in mice or glutathione S-transferase pi in humans. In one embodiment, the amount of one or more of glutathione S-transferases in a biological sample is determined by measuring the differential levels of expression of various isoenzymes of glutathione S-transferase, while in another aspect of this invention, the expression of glutathione S-transferase pi2 can be determined. In yet another embodiment of this invention the amount of one or more glutathione S-transferases can be directly measured. In another embodiment of this invention the amount of one or more glutathione S-transferases can be determined by measuring enzymatic activity corresponding to one or more glutathione S-transferases.

For example, the level of protein expression can be assessed by detecting the mRNA corresponding to the protein or proteins interest. More specifically, a biological sample can be divided into two equal halves. One half is used to determine the first amount of one or more glutathione S-transferases which can be referred to as a “control sample” while the other half is used to determine the second amount one or more of glutathione S-transferases can be referred to as a “test sample”. As used herein the term biological sample means a sample of metabolically active tissue or fluid containing cells in it. In the preferred embodiment, the biological sample is plasma, brain tissue or urine. No special preparation is made from these samples, although prior to exposure to a putative toxin to activate the glutathione S-transferase pi in cells, it might be necessary to concentrate the cellular material in each of the samples. Cells from the control sample are isolated and mRNA from these cells are isolated. Once mRNA is purified, the expression levels of the one or more glutathione S-transferases is determined using PCR primers. The mRNA is isolated from the test and control samples and complementary cDNA is prepared from the isolated mRNA. Using primers for the one or more glutathione S-transferases, the cDNA is amplified. Mouse primers are set out as sequence ID NOS. 5-27, while various preferred human primers are set out in SEQ ID NOS. 29-31, 33-35, 37-39, 41-43, 45-47, 49-51, 53-55, 57-59, 61-63, 65-67, and 69-71. The resultant amplification products are quantified as glutathione S-transferases gene sequences. The determined mRNA levels for the control sample are considered the baseline glutathione S-transferase levels.

In an alternate method to detect mRNA levels as disclosed by Sutcliffe, et al. U.S. Pat. No. 6,110,680, wherein cDNA is formed using anchor primers to fix a 3′-end-point. Cloned inserts are produced from the cDNA in a vector containing a bacteriophage specific promoter for subsequent RNA synthesis, linearized fragments of the cloned inserts are generated, cRNA is prepared, cDNA from the cRNA is transcribed using a set of primers and PCR is performed using 3′-primer whose sequence is derived from the vector and set of 5′-primers.

The amount of glutathione S-transferase, or one or more of its isoenzymes, in a biological sample can also be determined by radioimmunoassays, enzyme immunoassays or immunoflurometric assays (Tiainen P., et al. Clin. Chem. 42:334-35 (1996)); Doyle, et al., U.S. Pat. Nos. 6,080,551 and 6,183,977. Similarly, the amount of glutathione S-transferases can be determined by measuring enzymatic activity corresponding to one or more glutathione S-transferases. For example, Mulder et al., in Measurement of Glutathione S-transferase pi-1 in Plasma 80 Cancer 873 (1997) (hereby incorporated by reference) describes at pages 874-875 an elisa procedure, purification and preparation of GST pi-1 antibodies and a sample collection procedure. Additionally, Immun Diagnostik provides a kit for measuring GST-pi in humans (Immuno Diagnostik Order No. K7960).

The next step of the method involves contacting a biological sample with the environmental toxin. The test sample will then be exposed to an environmental toxin such as 1-methyl-4-phenyl-1, 2, 3, 6-tetrahydropyridine (MPTP) or a structurally related compound (for example, rotenone or paraquat; additionally, the toxin can be defined as a toxin or metabolite that interferes with Complex I respiration in a cell's electron transport chain.) MPTP structurally resembles a number of known environmental agents, including well-known herbicides such as paraquat (Di Monte, D., et al., Comparative Studies on the Mechanisms of Paraquat and 1-methyl-4-phenylpyridine (MPP+) Cytotoxicity, Biochem. Biophys. Res. Commun., 137:303-09 (1986)) and garden insecticides/fish toxins, such as rotenone (McNaught, K. S., et al., Effects of Isoquinoline Derivatives Structurally Related to 1-methyl-4-phenyl-1,2,3,6-tetrahydropyridine (MPTP) on Mitochondrial Respiration, Biochem. Pharmacol., 51:1503-11 (1996)) that have been shown to induce dopamine cell degeneration (Brooks, A. I., et al., Paraquat Elicited Neurobehavioral Syndrome Caused by Dopaminergic Neuron Loss, Brain Res., 823:1-10 (1999)).

MPTP is a lipophillic molecule that rapidly enters the brain and is taken-up into glial cells by a number of mechanisms including monoamine (Brooks W J, et al., Astrocytes as a Primary Locus for the Conversion MPTP into MPP+, J. Neural. Transm. 76:1-12 (1989)) and glutamate (Hazell A S, et al., 1-Methyl-4-phenyl-1,2,3,6-tetrahydropyridine (MPTP) Decreases Glutamate Uptake in Cultured Astrocytes, J. Neurochem. 68:2216-19 (1997)) transporters or pH-dependent antiporters (Kopin I J, Features of the Dopaminergic Neurotoxin MPTP, Ann NY Acad. Sci. 648:96-04 (1992)). Once in glial cells, MPTP is metabolized by the enzyme MAO-B to the unstable 1-methyl-4-phenyl-2,3-dihydropyridium (MPP+) which then rehydrogenates or deprotonates to generate MPTP or the corresponding pyridium species, MPP+, respectively. MPP+is then released from glia and taken up by neuronal dopamine transporters where it interferes with Complex I respiration in the electron transport chain (Nicklas W J, et al., MPTP, MPP+ and Mitochondrial Function, Life Sci. 40:721-29 (1987)). MPP+also binds to neuromelanin which could contribute to its neurotoxicity (D'Amato R J, et al., Selectivity of the Parkinsonian Neurotoxin MPTP: Toxic Metabolite MPP+ Binds to Neuromelanin, Sci. 231:987-89 (1986)). A recent report from our lab, using in vitro chimeric cell cultures, has demonstrated that the toxicity of MPTP is determined by the response of the glial cells following drug intoxication (Smeyne M, et al., Strain-Dependent Susceptibility to MPTP and MPP+-Induced Parkinsonism is Determined by Glia, Glia 74:73-80 (2001)). This is supported by numerous in vitro studies (Di Monte D A, et al., Production and Disposition of 1-methyl-4-phenylpyridinium in Primary Cultures of Mouse Astrocytes, Glia 5:48-55 (1992)).

The biological sample and environmental toxin are contacted, for example at 37 degrees Celsius, for a predetermined amount of time, after which cells in the biological sample are isolated and a second amount of one or more glutathione S-transferases is determined. The second amount of one or more glutathione S-transferases can be determined according to any of the methods previously discussed for the first amount of one or more glutathione S-transferases.

If the second amount of one or more glutathione S-transferases is lower than the first amount of one or more glutathione S-transferases, then this indicates the subject has a higher level of susceptibility than a subject having the second amount of one or more glutathione S-transferases higher than the first amount of one or more glutathione S-transferases. Similarly, if the second amount of one or more glutathione S-transferases is higher than the first amount of one or more glutathione S-transferases, this indicates a subject having a lower level of susceptibility than a subject having a second amount of one or more glutathione S-transferases lower than that or similar to the first amount glutathione S-transferase.

Similarly, if the second amount of one or more glutathione S-transferases is lower than the first amount of one or more glutathione S-transferases then this indicates a higher risk of the subject developing Parkinson's disease relative to a subject having a second amount of one or more glutathione S-transferases lower than or similar to the first amount of one or more glutathione S-transferases.

By examining levels of one or more glutathione S-transferases, and more specifically glutathione S-transferase pi in humans, one can identify at risk individuals prior to the onset of Parkinson's disease.

In another aspect of this invention, a method to determine the risk of development of idiopathic Parkinson's disease, is provided involving a method to screen biological samples for a genetic defect in genes encoding glutathione S-transferase pi proteins. More specifically, nucleic acid probes useful for the detection of genetic defects in genes encoding glutathione S-transferase pi can be prepared. The probe can be produced from an isolated whole chromosome nucleic acids found in the genes encoding glutathione S-transferase pi. Methods of preparing probes are well known to those skilled in the art (see, e.g. Sambrook et al. Molecular Cloning: A Laboratory Manual (2nd ed.) Vols 1-3, Cold Spring Harbor Laboratory, (1989) or Current Protocols in Molecular Biology, F. Ausubel et al. ed. Green Publishing and Wiley-Interscience, New York (1987)).

The term “probe,” as used herein means one or more nucleic acid fragments whose hybridization to a target sequence can be detected. The probe is labeled so that upon binding to the target sequence, it can be detected. Methods of labeling nucleic acids are well known to those skilled in the art. The term “hybridizing” refers to binding two single stranded nucleic acids via complementary base pairing. “Bind(s) specifically” refers to complementary hybridization between an oligonucleotide probe and a target sequence. The term “hybridization complex” refers to the complex of the probe the target sequence. The term “target sequence” refers to the nucleic acid to be detected or determined. A biological sample can be taken from a subject. In this embodiment, a biological sample is prepared by depositing cells, either as single cell suspensions or as tissue preparation, on solid supports such as glass slides and fixed by choosing a fixative which provides the best spatial resolution of the cells and the optimal hybridization efficiency. A plurality of labeled probes that bind specifically to a portion of the gene that encodes to glutathione S-transferase pi are added to the biological sample under hybridizing conditions. One skilled in the art will appreciate that the probes of this invention need not be absolutely specific for the targeted gene, glutathione S-transferase pi. Rather, in certain embodiments the probes are intended to produce staining contrast.

In one embodiment, the glutathione S-transferase pi2 gene is detected through the hybridization of probes of this invention to a target nucleic acid (e.g., a chromosome sample) in which it is desired to screen for the glutathione S-transferase pi gene. Other methods known to those skilled in the art include southern blots, in situ hybridization and quantitative amplification methods using primers, such as PCR (Innis et al., PCT Protocols, A Guide to Methods and Applications, Academic Press, Inc. (New York, 1990)).

In another aspect of this invention a method to determine the genetic susceptibility to an environment toxin is provided. This method involves contacting a biological sample from a subject with one or more probes each of which binds selectively to a region on mouse chromosome 1 spanning a 20 cM regions from D1Mit113 to D1Mit293. In the preferred embodiment the biological sample can be a chromosome sample from a mouse. The mouse sequences can be used to determine if any compound for human use has the potential to activate this system and therefore increase the risk for development of meurodegenerative disorders such as Parkinson's disease. The probe and chromosomal sample are combined under conditions to form a stable hybridization complex with the target sequence. The hybridization complex can be detected via the label on the probes.

In another genetic screen contemplated by this invention a biological sample from a human subject is contacted with probes that bind selectively to a segment of human GSTP1 (chr11:69874218-69877056); GST mu (hGSTm4: chr1:110677671-110687021, hGSTm2: chr1:110689771-110696957, hGSTm3: chr1:110758175-110761973, hGSTm1:chr1:110709530-110715415; or GST alpha (hGSTa1:chr6:52658645-52670705, hGSTa2:chr6:52617245-52630341; hGSTa3:chr6:52763514-52776547, hGSTa4:chr6:52844814-52862163. In the preferred embodiment the biological sample can be a chromosomal sample from man. The probe and chromosomal sample are combined under conditions to form a stable hybridization complex with the target sequence. The hybridization complex can be detected via the label on the probes. The detected hybridization complex is indicative of an increased susceptibility of a subject to develop Parkinson's.

This invention also provides a method for determining the susceptibility of a subject to develop Parkinson's disease. More specifically, this invention provides a method to determine the susceptibility to develop Parkinson's disease involving the steps of: contacting a biological sample from a subject with a composition made of a plurality of labeled probes each of which selectively binds to a region of human Glutathione S-transferase pi within a segment of human GSTP1 (chr11:69874218-69877056); GST mu (hGSTm4: chr1:110677671-110687021, hGSTm2: chr1:110689771-110696957, hGSTm3: chr1:110758175-110761973, hGSTm1:chr1:110709530-110715415; or GST alpha (hGSTa1:chr6:52658645-52670705, hGSTa2:chr6:52617245-52630341; hGSTa3:chr6:52763514-52776547, hGSTa4:chr6: 52844814-52862163 under conditions which a plurality of labeled probes form stable hybridization complexes with a segment of human GSTP1 (chr11:69874218-69877056); GST mu (hGSTm4: chr1:110677671-110687021, hGSTm2: chr1:110689771-110696957, hGSTm3: chr1:110758175-110761973, hGSTm1:chr1:110709530-110715415; or GST alpha (hGSTa1:chr6:52658645-52670705, hGSTa2:chr6:52617245-52630341; hGSTa3:chr6:52763514-52776547, hGSTa4:chr6:52844814-52862163; and detecting the hybridization complex wherein the hybridization complex is indicative of increased susceptibility of a subject to develop Parkinson's disease. Additionally, this invention involves the determination of the presence or absence of an allele of a polymorphic marker in the DNA of a subject. “Polymorphism” refers to the occurrence of two or more genetically determined alternative sequences or alleles in a population. A polymorphic marker is the locus at which divergence occurs. Preferred markers have at least two alleles, each occurring at a frequency of greater than 1%. A polymorphic locus may be as small as one base pair. More specifically, this invention provides methods of identifying subjects having a variant allele of a gene with a phenotype associated with the increased likelihood of development of Parkinson's disease. The gene is within a segment of human GSTP1 (chr11:69874218-69877056); GST mu (hGSTm4: chr1:110677671-110687021, hGSTm2: chr1:110689771-110696957, hGSTm3: chr1:10758175-110761973, hGSTm1:chr1:110709530-110715415; or GST alpha (hGSTa1:chr6:52658645-52670705, hGSTa2:chr6:52617245-52630341; hGSTa3:chr6:52763514-52776547, hGSTa4:chr6:52844814-52862163. An allele of the gene present in a person not suffering from Parkinson's disease is designated as wildtype. A variant allele of the gene is associated with a phenotype of an increased likelihood of developing Parkinson's disease in that an addition, deletion or substitution of nucleotides relative to the wildtype allele cause a phenotype of increase likelihood of Parkinson's disease. This phenotype may result from a nucleotide change in the gene affecting the expression of the gene or the nature of the resulting expression product, e.g., glutathione S-transferase.

One skilled in the art can determine linkage between a polymorphic marker and a locus associated with a particular phenotype is performed by mapping polymorphic markers by observing whether they co-segregate with the phenotype on a chromosome in an informative meiosis. (See, e.g., Kerem et al., Science 245:1073-80 (1989)). More specifically, linkage may be established by an affected sib-pairs analysis described by Terwilliger and Ott, Handbook of Human Genetic Linkage (Johns Hopkins, Md. 1994, ch. 06).

### EXAMPLE 1

### Expression of Gst Family Members in C57b1/6 and Swiss-Webster Mice

Studies have shown that different members of the glutathione S-transferase family (pi, alpha and mu) are differentially expressed throughout the body (Johnson J A, el Barbary A, Kornguth S E, Brugge J F, Siegel F L, Glutathione S-transferase Isoenzymes in Rat Brain Neurons and Glia, J. Neurosci. 13:2013-23 (1993)) although at this time no information has been published on the expression of these genes in the substantia nigra. Northern analysis of glutathione S-transferase pla and plb and mu showed that these genes are expressed throughout the brain. The levels of GSTmu do not appear to be changed after administration of MPTP, while mRNA levels of GSTpi may be decreasing 40 minutes after administration of MPTP in C57B1/6 animals but not in SW animals.

Higuchi et al (Higuchi R, et al., Simultaneous Amplification and Detection of Specific DNA Sequences, Biotech. (NY) 10:413-17 (1992)) pioneered the analysis of PCR kinetics by constructing a system that detects PCR products as they accumulate. This “real-time” system includes the intercalator ethidium bromide in each amplification reaction, an adapted thermal cycler to irradiate the samples with ultraviolet light, and detection of the resulting fluorescence with a computer-controlled cooled CCD camera. mRNA amplification produces increasing amounts of double-stranded DNA, which binds ethidium bromide, resulting in an increase in fluorescence. By plotting the increase in fluorescence versus cycle number, this method allows one to make precise quantitative measurements of mRNA expression. A single real time PCR analysis of glutathione S-transferase pi2 expression was performed to examine baseline levels of glutathione S-transferase pi2 in C57B1/6 and SW mice. Results of this experiment showed that baseline levels glutathione S-transferase pi2 from substantia nigra of C57B1/6 were similar to the levels seen in Swiss-Webster mice. Forty eight hours after MPTP administration, we detected a 50% lower induction of this gene in C57B1/6 mice compared to Swiss-Webster. Prior studies have shown that the GST genes are rapidly induced, some in as little as 8 hours following toxin exposure. The levels of these detoxification enzymes are then fairly rapidly returned to baseline levels. This suggests that our 50% reduction at forty eight hours will be much more significant when examined at earlier times.

Strain differences in response to MPTP administration can be recapitulated in vitro.

We have developed a procedure that allows us to culture postnatal substantia nigra (Smeyne M, et al., Method for Culturing Postnatal Substantia Nigra as an In Vitro Model of Experimental Parkinson's Disease, Brain Res. Proto. 9:105-11 (2002)). This was necessary since cultures generated from embryonic midbrain (a standard technique) contains numerous MPTP-resistant dopaminergic and noradrenergic neurons (Schneider J S, et al., Selective Loss of Subpopulations of Ventral Mesencephalic Dopaminergic Neurons in the Monkey Following Exposure to MPTP, Brain Res. 411:144-50 (1987)). When MPTP was administered to neurons in these cultures, we found little cell death in both the C57B1/6 and Swiss-Webster cultures (Smeyne M, et al., Strain-Dependent Susceptibility to MPTP and MPP+-Induced Parkinsonism is Determined by Glia, Glia 74:73-80 (2001)). In order to enrich for SN neurons, we generated cultures from postnatal animals in which the ventral midbrain populations had fully migrated to their final positions (Bayer S A, et al., Time of Neuron Origin and Gradients of Neurogenesis in Midbrain Dopaminergic Neurons in the Mouse, Exper. Brain. Res. 105:191-99 (1995)). On the basis of cell counts of these areas (Baker H, et al., Genetic Control of Number of Midbrain Dopaminergic Neurons in Inbred Strains of Mice: Relationship to Size and Neuronal Density of the Striatum, Proc. Nat. Acad. of Sci. (USA) 77:4369-73 (1980)), we estimated that our cultures were enriched 3-to-6 fold for SN neurons compared to whole midbrain preparations. When we examined cell loss in the SN cultures generated in parallel from both strains of mice, we observed that C57B1/6 mice had a loss of 50±5% of T-positive neurons while cultures derived from SWR mice showed a loss of 10±3%. The percent cell loss seen in our in vitro system was approximately 80% of the in vivo value. The 20% difference was likely due the presence of residual MPTP-resistant cell populations that could not be separated during the ventrolateral dissection. In addition, our strain-specific cell loss was observed using a concentration of MPTP that was 200-fold less (50×10-9 vs. 10×10-6) than other studies that examined the effects of MPTP in vitro (Sanchez-Ramos J R, et al., Selective Destruction of Cultured Dopaminergic Neurons From Fetal Rat Mesencephalon By 1-methyl-4-phenylpyridinium: Cytochemical and Morphological Evidence, J. Neurochem. 50:1934-44 (1988)). It is likely that the toxicity seen using lower concentrations of MPTP than previous studies was due to the enrichment of substantia nigra neurons (German D C, et al., The Neurotoxin MPTP Causes Degeneration of Specific Nucleus A8, A9 and A10 Dopaminergic Neurons in the Mouse, Neurodegeneration 5:299-312 (1996)) versus other catacholaminergic neurons present in whole midbrain preparations that have been shown to be less sensitive to MPTP.

Materials and Methods

Animals

C57BL/6J (Jackson Laboratory), Swiss-Webster (SWR, Harlan) and C57BL/6J×SWR used in these experiments were between 3 and 5 months of age. The F1 animals were derived both from male C57BL/6J×female SWR crosses as well as from female C57BL/6J×male SWR crosses. These animals were backcrossed to SWR to make a set of backcross (N2) progeny that were used to map chromosomal regions that underlie the large strain differences in MPTP neurotoxic response. All animals were generated within our vivarium at St. Jude Children's Hospital and were maintained on a 12:12 hour light: dark cycle with ad libitum food and water. Animals were treated in accord with Animal Care and Use Committee requirements. The analysis presented here is based on neuroanatomical and morphometric data from 58 animals, all of which were genotyped as described below.

MPTP Treatment

1-methyl-4-phenyl-1,2,3,6-tetrahydropyridine (MPTP, Sigma-St. Louis, Mo.) was dissolved in sterile saline to a final concentration of 4 mg/ml. Each animal was given a total of 80 mg/kg of MPTP, using a dosage regimen of 4 equal subcutaneous injections of 20 mg/kg, one given every 2 hours. Mice were monitored over the next 24 hours for the degree of mortality and the presence of seizures. All mice that survived the injection protocol were sacrificed one week after MPTP administration.

Histology

Mice were anesthetized with an overdose of Avertin. Following induction of deep anesthesia, animals were intracardially perfused with physiologic saline followed by 4% paraformaldehyde in 1× phosphate-buffered saline (PBS), pH 7.4. Brains were dissected out of the skull, post-fixed overnight in fresh fixative, dehydrated through a graded series of ethanols, defatted in mixed xylenes and embedded in Paraplast-X-tratm (Fisher Scientific). Brains were subsequently blocked and serially sectioned at 5 mm in the coronal plane. All sections from the rostral hippocampus to the cerebellar-midbrain junction was saved and mounted onto Superfrost-Plus slides (Fisher Scientific).

Immunocytochemistry

Standard tyrosine hydroxylase (TH) immunocytochemistry was performed as previously described (Hamre, K., et al., Differential Strain Susceptibility Following 1-methyl-4-phenyl-1,2,3,6-tetrahydropyridine (MPTP) Administration Acts in an Autosomal Dominant Fashion: Quantitative Analysis in Seven Strains of Mus Musculus, Brain Res., 828: 91-103 (1999)). The primary antibody was obtained from Pelfreeze (Rogers, A R) and used at a dilution of 1:1000. Incubation with the primary antibody was conducted overnight at RT. Standard avidin-biotin immunocytochemistry was conducted using the Vectastain ABC rabbit kit (Vector Laboratories). Immunolabeling was visualized using 3.3° diaminobenzidine as the chromagen. All sections were counter-stained with the Nissl stains cresyl violet or neutral red to insure that any decrease in cell numbers was not simply due to a decrease in tyrosine hydroxylase activity. All slides were subsequently dehydrated through ethanols, cleared in xylenes and coverslips were applied with Permount.

Cell Counting

Prior to counting TH-positive cells, the rostral to caudal boundaries of the SNpc were identified (German, D. C., et al., The Neurotoxin MPTP Causes Degeneration of Specific Nucleus A8, A9 and A10 Dopaminergic Neurons in the Mouse, Neurodegeneration, 5:299-312 (1996)). Each microscope slide contained five 5 micron sections that sampled 25 microns of tissue. TH-positive cells within the SNpc in one section per slide were counted using a 20× objective (total magnification 200×). Cells were counted as present within a section if they exhibited DAB reaction product in the cytoplasm and there was a clear and complete nucleus with nucleolus present. Once sections in each measured structure were counted, we summed the cell totals, multiplied by 5 to correct for uncounted sections and corrected for split nuclei using a modified Abercrombie correction factor (Abercrombie, M., Estimation of Nuclear Population From Microtome Sections, Anat. Rec, 94:239-247 (1946)). The number of cells in the SNpc from several animals were also counted using unbiased stereological measurements using the Bioquant image analysis system (R & 1 Biometrics, Nashville, Tenn.). Comparison using a Student's t-test between the number of cells counted using each method showed no statistical difference in cell number.

Microsatellite Markers

To identify and map QTLs, we genotyped a set of polymorphic MIT microsatellite markers that differentiate C57BL/6 from SWR. While C57BL/6 mice have been typed at all of these microsatellite loci (Dietrich, W. F., et al., A Genetic Map of the Mouse With 4,006 Simple Sequence Length Polymorphisms, Nat. Genet., 7:220-45 (1994)), Swiss-Webster had not been as thoroughly typed. We therefore tested a set of 150 MIT microsatellite markers, of which 64 effectively differentiated between these two strains. We typically typed at least three markers per chromosome (Table 3).

TABLE 3
List of microsatellite markers used in QTL study

| Marker Chr. | cM | |
|---|---|---|
| D1Mit211 | 1 | 12.0 |
| D1Mit100 | 1 | 74.3 |
| D1Mit111 | 1 | 91.8 |
| D1Mit113 | 1 | 91.8 |
| D1Mit293 | 1 | 113.7 |
| D2Mit416 | 2 | 12.0 |
| D2Mit458 | 2 | 31.7 |
| D3Mit19 | 3 | 66.7 |
| D4Mit192 | 4 | 10.9 |
| D4Mit13 | 4 | 71.0 |
| D5Mit338 | 5 | 43.7 |
| D5Mit287 | 5 | 82.0 |
| D6Mit273 | 6 | 9.8 |
| D6Mit291 | 6 | 55.7 |
| D7Mit117 | 7 | 10.9 |
| D7Mit238 | 7 | 42.6 |
| D7Mit259 | 7 | 67.8 |
| D8Mit95 | 8 | 6.6 |
| D8Mit205 | 8 | 31.7 |
| D9Mit205 | 9 | 13.1 |
| D9Mit32 | 9 | 33.9 |
| D9Mit116 | 9 | 60.1 |
| D10Mit282 | 10 | 7.7 |
| D10Mit186 | 10 | 36.1 |
| D10Mit292 | 10 | 57.9 |
| D10Mit297 | 10 | 75.4 |
| D11Mit78 | 11 | 4.4 |
| D11Mit29 | 11 | 37.2 |
| D11Mit334 | 11 | 71.0 |
| D12Mit169 | 12 | 3.3 |
| D12Mit214 | 12 | 32.8 |
| D12Mit280 | 12 | 51.4 |
| D13Mit78 | 13 | 2.2 |
| D14Mit262 | 14 | 37.2 |
| D14Mit266 | 14 | 67.8 |
| D15Mit53 | 15 | 29.5 |
| D15Mit161 | 15 | 65.6 |
| D16Mit181 | 16 | 3.3 |
| D16Mit4 | 16 | 25.1 |
| D16Mit106 | 16 | 6.6 |
| D17Mit117 | 17 | 25.1 |
| D17Mit42 | 17 | 41.5 |
| D18Mit223 | 18 | 6.6 |
| D18Mit213 | 18 | 37.2 |
| D19Mit90 | 19 | 28.4 |
| DXMit166 | X | 55.7 |
| DXMit117 | X | 18.6 |
| DXMit186 | X | 42.36 |

Amplification of Microsatellite Markers

DNA samples from each individual and the control parental strains were amplified using PCR thermal cycling parameters described in detail at www.nervenet.org/papers/PCR.html. We used a touchdown PCR protocol to improve the specificity of annealing. The products were all run on Metaphor agarose, photographed, and scored manually. Data were entered into a relational database (FileMaker Pro).

Calculating Linkage Between Loci

We compared the distribution pattern of phenotypes of the mice (high or low SNpc number following MPTP treatment) with the distribution pattern of sensitive (C57BL/6J=B) and resistant (SWR=S) alleles at polymorphic microsatellite loci. The first level of analysis was simply to detect a linkage, whereas the second level involved estimating QTL position more precisely by interval mapping. Actual calculations were performed using the program Map Manager QTX b 15 that is freely available from Kenneth Manly and colleagues (Roswell Park Cancer Institute, Buffalo, N.Y.). This program is available on the WWW at: http://mapmgr.roswellpark.org/mmQTX.html.

Results

Cell Counts

58 animals survived the MPTP treatments and were used for the QTL analysis. Once the number of SNpc cells from each of these animals was counted (Table 4), the total number of SNpc cells was used to calculate a correlation to each microsatellite marker. In addition, animals were also determined to be C57B1/6 or SWR in appearance based upon a comparison of number of SNpc cells following MPTP treatment-essentially a Mendelian score scheme. Animals were assigned to the C57BL/6 category if the number of SNpc cells was less than 3300. The number of SNpc neurons in both of the parental lines prior to treatment are known to be very close to each other with a combined mean of 6518±230. Following MPTP treatment, the number of SNpc neurons in C57B1/6 animals was 3300±200, while the number of SNpc neurons in the SWR animals was 5480+/−239. A probability density map (essentially a smoothed histogram) of the cell counts for these 58 shows that the majority of cases had SNpc numbers similar to the C57B1/6 parental strain as predicted from our previous study that demonstrated that C57B1/6 sensitive alleles were dominant (Hamre, K., et al. Differential Strain Susceptibility Following 1-methyl-4-phenyl-1,2,3,6-idine (MPTP) Administration Acts in an Autosomal Dominant Fashion: Analysis in Seven Strains of Mus Musculus, Brain Res., 828: 91-103 (1999)).

TABLE 4
Raw data for QTL analysis

| Animal | Number of | Genotype |
|---|---|---|
| Number | SNpc cells | Assigned |
| 1 | 2373a | C57BL/6 |
| 2 | 2203 | C57BL/6 |
| 4 | 4435b | SWR |
| 5 | 3342 | SWR |
| 9 | 4378 | SWR |
| 16 | 1417 | C57BL/6 |
| 26 | 2101 | C57BL/6 |
| 29 | 2127 | C57BL/6 |
| 32 | 1381 | C57BL/6 |
| 33 | 4324 | SWR |
| 41 | 2657 | C57BL/6 |
| 43 | 4059 | SWR |
| 45 | 3371 | SWR |
| 48 | 3726 | SWR |
| 49 | 1992 | C57BL/6 |
| 51 | 3345 | SWR |
| 52 | 5113 | SWR |
| 57 | 3683 | SWR |
| 67 | 2021 | C57BL/6 |
| 76 | 4444 | SWR |
| 77 | 6198 | SWR |
| 81 | 3122 | C57BL/6 |
| 82 | 2619 | C57B1/6 |
| 83 | 4712 | SWR |
| 89 | 3710 | SWR |
| 91 | 3169 | SWR |
| 93 | 4316 | SWR |
| 94 | 4714 | SWR |
| 96 | 2599 | C57B1/6 |
| 111 | 2625 | C57BL/6 |
| 115 | 2479 | C57BL/6 |
| 118 | 3058 | C57BL/6 |
| 126 | 4871 | SWR |
| 127 | 3591 | SWR |
| 132 | 4115 | SWR |
| 133 | 1135 | C57B1/6 |
| 134 | 2232 | C57BL/6 |
| 135 | 2506 | C57BL/6 |
| 137 | 2884 | C57BL/6 |
| 140 | 5235 | SWR |
| 166 | 3434 | SWR |
| 169 | 1516 | C57BL/6 |
| | 4284 | SWR |
| 172 | 2063 | C57BL/6 |
| | 1926 | C57BL/6 |
| | 6286 | SWR |
| | 1889 | C57BL/6 |
| 182 | 2244 | C57BL/6 |
| | 2834 | C57BL/6 |
| | 4055 | SWR |
| 185 | 2233 | C57BL/6 |
| 186 | 3832 | SWR |
| | 1705 | C57BL/6 |
| | 3411 | SWR |
| | 4999 | SWR |
| | 4481 | SWR |
| | 3466 | SWR |
| 215 | 2866 | C57BL/6 |

a Animals were assigned to C57BL/6 if less than 3300 cell following MPTP treatment

b Animals were assigned to SWR if greater than 3300 cells following MPTP treatment

Mapping Variance in MPTP Dopaminergic Neuron Survival

Simple Interval Mapping

The best linkage was discovered on Chromosome 1. The LOD reaches a peak of 4.5 on Chromosome 1 between D1Mit113 at 173 Mb and D1Mit361 at 187 Mb. The probability of linkage was assessed by permuting phenotypes among progeny and remapping these permuted data sets. The genome-wide significance thresholds (significant and highly significant) (Doerge, R. W. et al., Permutation Tests for Multiple Loci Affecting a Quantitative Character, Genetics, 142:285-94 (1996)) were estimated to be near LOD scores of 2.8 and 4.9, respectively. The genome-wide probability associated with the peak LOD of 4.5 is approximately 0.005. The 2 LOD support interval extends from a proximal border at 144 Mb to the telomere at ˜195 Mb. This locus on Chromosome 1 that we have given the name MPTP sensitivity 1 (Mptp1) accounts for approximately 30% of the phenotypic variance.

Polarity of Effects

As shown in our previous analysis of parental strains and their F1 hybrids (Hamre, K., et al., Differential Strain Susceptibility Following 1-methyl-4-phenyl-1,2,3,6-tetrahydropyridine (MPTP) Administration Acts in an Autosomal Dominant Fashion: Quantitative Analysis in Seven Strains of Mus Musculus, Brain Res., 828:91-103 (1999)), the unusually high MPTP vulnerability of dopaminergic neurons in C57BL/6 is dominant over the resistant phenotype typified by several Swiss Webster strains. We therefore expected the C57BL/6 allele at the major QTL on Chromosome 1, Mptp1, to be associated with high MPTP sensitivity and increased TH cell loss. In fact, the observed pattern is opposite to this expectation. The inclusion of a single allele inherited from C57BL/6 at this locus results in a distinct positive effect and TH-positive cells in the SN are spared in comparison to mice that are SWR homozygotes. Conversely, homozygosity of SWR alleles contribute to a loss of approximately 1400 cells. The correlation between TH-positive neuron number after MPTP treatment and genotypes (SS scored as 1 and BS heterozygotes scored as 0) is approximately −0.55. This surprising finding leaves open the question of the genomic location of loci (either a single locus or several loci associated through epistatic interactions) that contribute to increased sensitivity in C57BL/6.

Composite Interval Mapping

In addition experiments for two markers that flank Mptp1 (D1Mit113 and D1Mit361) were controlled and used composite interval mapping to search for other loci that may have additive effects that explain the differential sensitivity to MPTP between the two parental strains. There is no indication of any strong additive secondary loci and the highest LOD score after controlling for Mptp1 is well under 1.0.

Analysis of Potential Epistatic Interactions

Two-way epistatic interactions were investigated using Map Manager QTX b15, while both controlling and not controlling for Mptp1 (flanking markers D1Mit113 and D1Mit361). When Mptpt1 was not controlled, we detected no significant epistatic interactions. With composite control for Mptp1 several pairs of markers had nominal interaction terms with probabilities of approximately p≦0.001. These highest statistical interactions occurred between D7Mit259 and D12Mit214, D16Mit181 and D17Mit117, D8Mit121 and D12Mit214, and D11Mit334 and D14Mit206. Subsequent statistical analysis of the polarity of these interaction effects did not produce any locus combinations with the expected polarity (SWR alleles protective and B6 alleles sensitizing). Comparable analysis without control for Mptp1 did not reveal any significant epistatic interactions.

Discussion

In this invention we have identified a region on mouse chromosome 1, spanning a 20 cM region from D1Mit113 to D1Mit293. Unexpectedly we found that the correlation between C57BL/6 and cell death was negative. This suggests to us that the increased cell death seen in the C57BL/6 versus SWR animals is due to a decrease in a protective factor, rather than an increase in a factor that induces or directly regulates the induction of cell death. Previously, using an in vitro assay (Smeyne, M., et al., Strain-Dependent Susceptibility to MPTP and MPP+-Induced Parkinsonism is Determined by Glia, Glia, 74:73-80 (2001)) we have demonstrated the primacy of the glial cell as a main determinate for MPTP-induced cell loss; i.e. C57BL/6 neurons could be rescued from MPTP toxicity by growth on SWR glia. The correlational data presented in this study supports our hypothesis that the glia provide a protective factor that is reduced in C57BL/6 mice compared to SWR mice.

Most of the sequence in the mptp1 QTL region of chromosome 1 is marked by expressed sequence tags rather than identified genes. Based upon sequences and gene discovery on the UCSC genome bioinformatics site (http://genome.ucsc.edu/), 66 known genes are contained within this QTL (chr1:base pairs: 172988033-194630025). Of these 66, 29 are expressed in significant amounts in mRNA isolated from C57BL/6 whole brain (minus olfactory bulb, cerebellum and brainstem). In terms of the known pathophysiology of Parkinson's disease, only a few of these stand out as possible Parkinson's disease candidate genes. These include the potassium channel, subfamily K, member 2, Kcnk2 (Bockenhauer, D., et al., KCNK2: Reversible Conversion of a Hippocampal Potassium Leak into a Voltage-Dependent Channel, Nat. Neurosci., 4:486-91 (2001)), calpain 2, Capn2, presenelin 2, Psen2, and potassium inwardly-rectifying channel, subfamily, Kcnj9 which is also known as Kir3.3 or GIRK3. Kcnj3 is of particular interest because is expressed in the SNpc and another family member, GIRK2 when mutated has been shown to lead to SNpc degeneration.

In addition to these potential major effect genes, we also have detected several chromosomal regions that may harbor genes that interact in non-linear ways with Mptp1. Epistasis is defined as a non-additive effect of alleles at one locus on those at another locus. The epistatic regions may provide a few clues on the cause of MPTP-induced strain susceptibility to cell loss. Several of these epistatic regions contains genes known to affect cell death as well as several members of the inward rectifying potassium channels.

### EXAMPLE 2

### Sequencing of the GSTpi2 Gene

In this invention we searched the Ensembl and Celera mouse genomic databases using GSTp1 cDNA as template. A putative transcript was identified on mouse Chromosome 1 (shown below as Query) that shows significant homology to the existing GSTp1 cDNA clones located on mChr 19 (Shown below as SJBCT, SEQ. ID NO. 4) (Xu and Stambrook, 1994).

TABLE 5

| | | | |
|---|---|---|---|
| Query: | 1 | atgccgccgtacaccattgtctacttcccaagtcgagggcggtgtgaggccatgcgcatg | 60 |
| | | \|\|\|\|\| \|\| \|\|\|\|\|\|\|\|\|\|\|\|\|\|\|\|\|\|\|\|\| \|\|\|\|\|\|\|\|\|\|\|\|\|\|\|\|\|\|\|\|\|\|\|\| \|\|\| | |
| Sbjct: | 55 | atgccaccatacaccattgtctacttcccagttcgagggcggtgtgaggccatgcgaatg | 114 |
| Query: | 61 | ctgctgactgaccagggccagagctggaaggaggaggtggttaccatagatacctggatg | 120 |
| | | \|\|\|\|\|\| \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 115 | ctgctggctgaccagggccagagctggaaggaggaggtggttaccatagatacctggatg | 174 |
| Query: | 121 | caaggcttgctcaagcccacttgtctgtatgggcagcttcctaagtttgaggatggagac | 180 |
| | | \|\|\|\|\|\|\|\|\|\|\|\|\|\|\|\|\|\|\|\|\|\|\|\|\|\|\|\|\|\|\|\|\|\|\|\|\|\| \|\| \|\|\|\|\|\|\|\|\|\|\|\|\|\|\|\|\|\| | |
| Sbjct: | 175 | caaggcttgctcaagcccacttgtctgtatgggcagctccccaagtttgaggatggagac | 234 |
| Query: | 181 | ctcaccctttaccaatctaatgccatcttgagacaccttggccgctctttggggctttat | 240 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 235 | ctcaccctttaccaatctaatgccatcttgagacaccttggccgctctttggggctttat | 294 |
| Query: | 241 | gggaaaaaccagagggaggccgcccaggtggatatggtgaatgatggggtagaggacctt | 300 |
| | | \|\|\|\|\|\|\|\|\|\|\|\|\|\|\|\|\|\|\|\|\|\|\|\|\|\|\| \|\|\|\|\|\|\|\|\|\|\|\|\|\|\|\|\|\|\|\|\|\| \|\|\|\|\|\|\|\|\| | |
| Sbjct: | 295 | gggaaaaaccagagggaggccgcccagatggatatggtgaatgatggggtggaggacctt | 354 |
| Query: | 301 | cgcggcaaatatggcaccatgatctacagaaactatgagaatggtaagaatgactacgtg | 360 |
| | | \|\|\|\|\|\|\|\|\|\|\|\|\| \|\|\|\| \| \|\|\|\|\|\|\| \|\|\|\|\|\|\|\|\|\|\|\|\|\|\|\|\|\|\|\|\|\|\|\|\|\|\|\|\|\| | |
| Sbjct: | 355 | cgcggcaaatatgtcaccctcatctacaccaactatgagaatggtaagaatgactacgtg | 414 |
| Query: | 361 | aaggccctgcctgggcatctgaagccttttgagaccctgctgtcccagaaccagggaggc | 420 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 415 | aaggccctgcctgggcatctgaagccttttgagaccctgctgtcccagaaccagggaggc | 474 |
| Query: | 421 | aaagctttcatcgtgggtgaccagatctcctttgccgattacaacttgctggacctgctg | 480 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 475 | aaagctttcatcgtgggtgaccagatctcctttgccgattacaacttgctggacctgctg | 534 |
| Query: | 481 | ctgatccaccaagtcctggcccctggctgcctggacaacttccccctgctctctgcctat | 540 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 535 | ctgatccaccaagtcctggcccctggctgcctggacaacttccccctgctctctgcctat | 594 |
| Query: | 541 | gtggctcgcctcagtgcccggcccaagatcaaggcctttctgtcctccccggaacatgtg | 600 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 595 | gtggctcgcctcagtgcccggcccaagatcaaggcctttctgtcctccccggaacatgtg | 654 |

| | | | | |
|---|---|---|---|---|
| Query: | 601 | aaccgtcccatcaatggcaatggcaaacag | 630 | (SEQ ID NO. 1) |
| | | \|\|\|\|\|\|\|\|\|\|\|\|\|\|\|\|\|\|\|\|\|\|\|\|\|\|\|\|\|\| | | |
| Sbjct: | 655 | aaccgtcccatcaatggcaatggcaaacag | 684 | (SEQ ID NO. 4) |

This novel transcript was predicted by the Ensembl analysis pipline from either a GeneWise or GeneScan prediction and subsequent confirmation of exons by comparisons to protein, cDNA and EST databases. The transcript contains a single exon of 630 bps on chromosome 1 (1.193000001-194000000 or ENSMUSE00000285985) (SEQ ID. NO.1). From the genomic sequence of this transcript, we designed two primers (5′-gcacttgccagcttcaggtgaattca-3′ (SEQ ID NO. 5) and 5′-gtcaggtagcttcaggcagaaccaca-3′ (SEQ ID NO. 6) flanking the entire exon and performed genomic PCR analysis on genomic DNA from both C57B1/6 and Swiss-Webster strains. The PCR conditions are standard and the annealing temperature is 58° C. The resulting product is about 800 bps based on the Ensembl database.

Real Time PCR

Higuchi et al (Higuchi R, et al., Simultaneous Amplification and Detection of Specific DNA Sequences, Biotechnology (N Y) 10:413-17 (1992)) pioneered the analysis of PCR kinetics by constructing a system that detects PCR products as they accumulate. This “real-time” system includes the intercalator ethidium bromide in each amplification reaction, an adapted thermal cycler to irradiate the samples with ultraviolet light, and detection of the resulting fluorescence with a computer-controlled cooled CCD camera. Amplification produces increasing amounts of double-stranded DNA, which binds ethidium bromide, resulting in an increase in fluorescence. By plotting the increase in fluorescence versus cycle number, this method produces amplification allows one to make precise quantitative measurements of mRNA expression.

We will be quantitating levels of glutathione S-transferase pi2 mRNA using this technique in place of traditional Northern blots since we have a limited amount of material to generate mRNA and real-time PCR amplification can give precise results with a small amount of sample material. Real time PCR is based on measurement of mRNA during the exponential phase of the reaction. Unlike traditional PCR methods, the Real-Time PCR uses 3 primers. A traditional 5′ and 3′ primer as well as a “labeled” primer that is generated between the 5′ and 3′ primer. Quantitative measurements are made based on the amount of fluorescence detected by amplification of the intervening primer. Details of this method are presented in numerous papers such as (Freeman W M, et al., Quantitative RT-PCR: Pitfalls and Potential. Biotechniques 26:112-122, 124-115 (1999)) as well as from the ABI website. (http://www.appliedbiosystems.com/molecularbiology/about/pcr/sds/). Primers for quantitative measurement of mRNA levels will be generated using software developed by ABI called Primer Express (Version 1.5) based upon nucleotide sequences imported from NCBI. The primers for measurement of the glutathione S-transferases are:

| | | |
|---|---|---|
| GSTpi2 | | |
| Ensembl gene | ENSMUSG00000037403 | |
| 5′ | CACCTGGGCCGCTCC | (SEQ ID NO. 7) |
| 3′ | TGTCCACCAGTGCTGCCTC | (SEQ ID NO. 8) |
| labeling primer | TCGGGCTCTATGGCAAAGACCAACA | (SEQ ID NO. 9) |
| GSTp1a/1b | | |
| Ensemble Gene | | |
| 5′ | TGTGAGGCCATGCGCA | (SEQ ID NO. 10) |
| 3′ | TGGTAACCACCTCCTCCTTCC | (SEQ ID NO. 11) |
| labeling primer | CTGCTGACTGACCAGGGCCAGAGC | (SEQ ID NO. 12) |
| GST alpha yc | | |
| Ensembl gene | ENSMUSG00000025934 | |
| 5′ | CAGGCTGAGCAGGGCTGA | (SEQ ID NO. 13) |
| 3′ | GCCCGGGTCCAGCTCTT | (SEQ ID NO. 14) |
| labeling primer | ATTGCCCTGGTTGAACTCCTCTACCATGTG | (SEQ ID NO. 15) |
| GST alpha GT41A | | |
| Ensembl gene | ENSMUSG00000032347 | |
| 5′ | GCAATGGCCGGGAAGC | (SEQ ID NO. 16) |
| 3′ | ACCTGATGCACTCCATTCTGC | (SEQ ID NO. 17) |
| labeling primer | CGTGCTTCACTACTTCAATGCCCGG | (SEQ ID NO. 18) |
| GST mu (chr 1) | | |
| Ensembl gene | ENSMUSG00000033859 | |
| 5′ | CAGGCACCTTATATTCGAACCC | (SEQ ID NO. 19) |
| 3′ | CGCAGGCCACAAAGTCCT | (SEQ ID NO. 20) |
| labeling primer | CGTGCCTGGATGCCTTCCTAAACCTG | (SEQ ID NO. 21) |
| GST mu 1 (chr 3) | | |
| Ensembl gene | ENSMUSG00000004035 | |
| 5′ | ATGCCATCCTGCGCTACC | (SEQ ID NO. 22) |
| 3′ | TCCTTTCCTCCTCTGTCTCCC | (SEQ ID NO. 23) |
| labeling primer | TGGCCGCAAGCACAACCTGTG | (SEQ ID NO. 24) |
| GST mu 2 (chr 18) | | |
| Ensembl gene | ENSMUSG00000041640 | |
| 5′ | CACCGAATATTTGAGCCCAAG | (SEQ ID NO. 25) |
| 3′ | CAAAGCGACCCATGAAGTCC | (SEQ ID NO. 26) |
| labeling primer | CCTGGACGCCTTCCCAAACCTGA | (SEQ ID NO. 27) |

All PCR reactions are performed using standard conditions that have been specifically calculated for the ABI PRISM 7700 system. Sequence-specific signal from the fluorogenic (intervening) primer is generated during the PCR. The ABI PRISM 7700 Sequence Detector provides the instrumentation for detecting this fluorescent signal during thermal cycling. The 7700 system has a built-in thermal cycler. The fluorescence emission travels through optic cables to a CCD camera detector. For each sample, the CCD camera collects the emission data between 520 nm and 660 nm once every few seconds. The system minimizes carryover contamination between samples because it monitors the fluorescent signal in sample tubes with closed optical caps and avoids the need to open the sample tubes. The software analyzes the data by first calculating the contribution of each component dye to the experimental spectrum. The reporter signal is then normalized to the fluorescence of an internal reference dye. Peak normalized reporter values are averaged for each cycle and plotted versus cycle number. The investigator chooses the slope at which to determine the amount of mRNA amplified. The PI-chosen point on the slope is accurate as long as measured in the exponential phase (cycles 12-22, empirically determined).

Western Blots

Animals will be decapitated, the SN dissected out on ice and snap frozen in liquid nitrogen and stored at −80° C. until biochemical analyses. The SN is homogenized in cold lysis buffer (300 ul/100 mg tissue) containing 250 mM NaCl, 50 mM tris (pH 7.5), 0.1% NP40, 1 mM EDTA, 20% glycerol, 5 mM dithiothreitol, 0.2 mM phenylmethylsulfonyl fluoride (PMSF), 10g/ml aprotinin, and leupeptin. The homogenate is centrifuged at 12,000×g for 10 min at 4° C. and the protein concentration of the supernatant determined by the Bradford assay using BSA as standard. A 50 ug aliquot of protein is separated by SDS/PAGE, transferred to nitrocellulose membranes (Amersham Pharmacia Biotech), blocked with 5% nonfat milk, washed with TBST (10 mM tris-HCL, pH 7.4/100 mM NaCl/O. 1% Tween 20) and incubated with primary antibodies overnight at 4° C. Primary antibodies to be used in these studies include: GST pi (Cat.#GSTP1-S, Alpha Diagnostic International, Inc. San Antonio, Tex.), alpha (Cat.#GSTA1-S, Alpha Diagnostic International, Inc. San Antonio, Tex.) and GST mu (Cat.#GSTM1-S, Alpha Diagnostic International, Inc. San Antonio, Tex.). After incubation of the individual primary antibodies, membranes are washed with TBST and incubated with appropriate secondary antibodies. Proteins are visualized by ECL (Amersham Pharmacia Biotech) and quantified by densitometry (Eagle Eye Detection System).

Immunocytochemistry

Glutathione S-transferase subtype specific immunohistochemistry will use the antibodies described above. SN neurons in culture will be visualized using antibodies directed against tyrosine hydroxylase (TH) (Sigma). Briefly, sections will be deparaffinized through mixed xylenes (2×5 minutes) and a descending series of ethanols (2×100% ETOH, 2×95% ETOH, 1×80% ETOH, 1×70% ETOH, 1×50% ETOH, 2×DH2O; each 3 minutes) until completely rehydrated. Following the rehydration, all antibody solutions will be prepared by mixed in phosphate-buffered saline (PBS, pH 7.4) with 0.2% Triton-X100 in 0.5% bovine serum albumin (Fraction V, Sigma). Washes, except for those following the avidin-biotin complex, will also be conducted in the same solution. The protocol for immunostaining starts with 2×5 minute washes in PBS, followed by a 30 minute blocking step in phosphate-buffered saline (PBS, pH 7.4) with 0.2% Triton-X100 in 0.5% bovine serum albumin (Fraction V, Sigma). Following the blocking step, sections are incubated with the primary antibody for 16 hours at 25° C. Visualization of the primary antibody is conducted using standard avidin-biotin immunocytochemistry utilizing the Vectastain ABC rabbit kit (Vector Laboratories). ABC complex is visualized using fluorescent secondary antibodies (FITC and RITC) as chromagens. For counting, SN neurons will be visualized using DAB as a chromogen. Following the immunostaining, all sections will be counterstained with the Nissl stains cresyl violet or neutral red to insure that any decrease in cell numbers is not simply due to a decrease in tyrosine hydroxylase activity. All slides will then be dehydrated through ethanols (1×DH20, 1×50% ETOH, 1 70% ETOH, 2×95% ETOH 2×1 00% ETOH; each step for 3 minutes), cleared in mixed xylenes (2×5 minutes) and coverslips applied with Permount. The whole of the immunostaining procedure will be done on a Leica Robot Immunostainer, Model #ST5050.

Similarly, the mRNA sequence of human glutathione transferases as known. For example, the mRNA sequence for GSTp1 is:

| hGSTp1 mRNA (NM_000852) | | | | | | | |
|---|---|---|---|---|---|---|---|
| 1 | ggagtttcgc | cgccgcagtc | ttcgccacca | tgccgcccta | caccgtggtc | tatttcccag | (SEQ ID NO: 28) |
| 61 | ttcgaggccg | ctgcgcggcc | ctgcgcatgc | tgctggcaga | tcagggccag | agctggaagg | |
| 121 | aggaggtggt | gaccgtggag | acgtggcagg | agggctcact | caaagcctcc | tgcctatacg | |
| 181 | ggcagctccc | caagttccag | gacggagacc | tcaccctgta | ccagtccaat | accatcctgc | |
| 241 | gtcacctggg | ccgcaccctt | gggctctatg | ggaaggacca | gcaggaggca | gccctggtgg | |
| 301 | acatggtgaa | tgacggcgtg | gaggacctcc | gctgcaaata | catctccctc | atctacacca | |
| 361 | actatgaggc | gggcaaggat | gactatgtga | aggcactgcc | cgggcaactg | aagccttttg | |
| 421 | agaccctgct | gtcccagaac | cagggaggca | agaccttcat | tgtgggagac | cagatctcct | |
| 481 | tcgctgacta | caacctgctg | gacttgctgc | tgatccatga | ggtcctagcc | cctggctgcc | |
| 541 | tggatgcgtt | ccccctgctc | tcagcatatg | tggggcgcct | cagcgcccgg | cccaagctca | |
| 601 | aggccttcct | ggcctcccct | gagtacgtga | acctccccat | caatggcaac | gggaaacagt | |
| 661 | gagggttggg | gggactctga | gcgggaggca | gagtttgcct | tcctttctcc | aggaccaata | |
| 721 | aaatttctaa | gagagct | | | | | |

From this sequence the forward primer, reverse primer and TaqMan primer can be identified using Primer Express® (Applied Biosystems). The primers shown below as SEQ ID NOS: 29-31 are preferred for the measurement of GSTp1, but other functional primers can be identified using SEQ ID NO: 28 and Primer Express® (Applied Biosystems) by one skilled in the art).

| | | |
|---|---|---|
| Forward primer: | (5′-3′):CCTGTACCAGTCCAATACCATCCT | (SEQ ID NO: 29) |
| Reverse primer: | (5′-3′):CCTGCTGGTCCTTCCCATAG | (SEQ ID NO: 30) |
| TaqMan primer: | (5′-3′):CGTCACCTGGGCCGCACCC | (SEQ ID NO: 31) |

| hGSta1 mRNA (NM_145740) | | | | | | | |
|---|---|---|---|---|---|---|---|
| 1 | aaatacatta | ggacctggaa | ttcagttgtc | gagccaggac | ggtgacagcg | tttaacaaag | (SEQ ID NO: 32) |
| 61 | cttagagaaa | cctccaggag | actgctatca | tggcagagaa | gcccaagctc | cactacttca | |
| 121 | atgcacgggg | cagaatggag | tccacccggt | ggctcctggc | tgcagctgga | gtagagtttg | |
| 181 | aagagaaatt | tataaaatct | gcagaagatt | tggacaagtt | aagaaatgat | ggatatttga | |
| 241 | tgttccagca | agtgccaatg | gttgagattg | atgggatgaa | gctggtgcag | accagagcca | |
| 301 | ttctcaacta | cattgccagc | aaatacaacc | tctatgggaa | agacataaag | gagagagccc | |
| 361 | tgattgatat | gtatatagaa | ggtatagcag | atttgggtga | aatgatcctc | cttctgcccg | |
| 421 | tatgtccacc | tgaggaaaaa | gatgccaagc | ttgccttgat | caaagagaaa | ataaaaaatc | |
| 481 | gctacttccc | tgcctttgaa | aaagtcttaa | agagccatgg | acaagactac | cttgttggca | |
| 541 | acaagctgag | ccgggctgac | attcatctgg | tggaacttct | ctactacgtc | gaggagcttg | |
| 601 | actccagtct | tatctccagc | ttccctctgc | tgaaggccct | gaaaaccaga | atcagcaacc | |
| 661 | tgcccacagt | gaagaagttt | ctacagcctg | gcagcccaag | gaagcctccc | atggatgaga | |
| 721 | aatctttaga | agaagcaagg | aagattttca | ggttttaata | acgcagtcat | ggaggccaag | |
| 781 | aacttgcaat | accaatgttc | taaagttttg | caacaataaa | gtactttacc | taa | |

From this sequence the forward primer, reverse primer and TaqMan primer can be identified using Primer Express® (Applied Biosystems). The primers shown below as SEQ ID NOS: 33-35 are preferred for the measurement of hGSTa1, but other functional primers can be identified using SEQ ID NO: 32 and Primer Express® (Applied Biosystems) by one skilled in the art).

| | | |
|---|---|---|
| Forward primer: | (5′-3′):AGAAACCTCCAGGAGACTGCTATC | (SEQ ID NO: 33) |
| Reverse primer: | (5′-3′):TGCCCCGTGCATTGAAG | (SEQ ID NO: 34) |
| TaqMan primer: | (5′-3′):TGGCAGAGAAGCCCAAGCTCCACT | (SEQ ID NO: 35) |

| hGSTa2 mRNA (NM_000846) | | | | | | | |
|---|---|---|---|---|---|---|---|
| 1 | tattgagagg | aacaaagagc | ttataaatac | attaggacct | ggaattcggt | tgtccagcca | (SEQ ID NO: 36) |
| 61 | caaaggtgac | agcatttaac | aaagcttaga | gaaacctcca | ggagactgct | accatggcag | |
| 121 | agaagcccaa | gctccactac | tccaatatac | ggggcagaat | ggagtccatc | cggtggctcc | |
| 181 | tggctgcagc | tggagtagag | tttgaagaga | aatttataaa | atctgcagaa | gatttggaca | |
| 241 | agttaagaaa | tgatggatat | ttgatgttcc | agcaagtgcc | aatggttgag | attgatggga | |
| 301 | tgaagctggt | gcagaccaga | gccattctca | actacattgc | cagcaaatac | aacctctatg | |
| 361 | ggaaagacat | aaaggagaaa | gccctgattg | atatgtatat | agaaggtata | gcagatttgg | |
| 421 | gtgaaatgat | ccttcttctg | ccctttactc | aacctgagga | acaagatgcc | aagcttgcct | |
| 481 | tgatccaaga | gaaaacaaaa | aatcgctact | tccctgcctt | tgaaaaagtc | ttaaagagcc | |
| 541 | acggacaaga | ctaccttgtt | ggcaacaagc | tgagccgggc | tgacattcac | ctggtggaac | |
| 601 | ttctctacta | cgtggaagag | cttgactcta | gccttatttc | cagcttccct | ctgctgaagg | |
| 661 | ccctgaaaac | cagaatcagt | aacctgccca | cagtgaagaa | gtttctacag | cctggcagcc | |
| 721 | caaggaagcc | tcccatggat | gagaaatctt | tagaagaatc | aaggaagatt | ttcaggtttt | |
| 781 | aataaaccag | ccatagaggt | caagaacatg | caagaccagt | attctaaagt | tttgcaacaa | |
| 841 | ttaagtgctt | tacctaagtg | ttgattgtgc | ctgttgtgaa | gctaatgaac | tctttcaaat | |
| 901 | tatatgctaa | ttaaataata | caactcctat | tcacccactt | agttaaaatt | gatttcttct | |
| 961 | cattaggatc | tgatgt | | | | | |

From this sequence the forward primer, reverse primer and TaqMan primer can be identified using Primer Express® (Applied Biosystems). The primers shown below as SEQ ID NOS: 37-39 are preferred for the measurement of hGSTa2, but other functional primers can be identified using SEQ ID NO: 36 and Primer Express®) (Applied Biosystems) by one skilled in the art).

| | | |
|---|---|---|
| Forward primer: | (5′-3′): GAGCCACGGACAAGACTACCTT | (SEQ ID NO: 37) |
| Reverse primer: | (5′-3′): AAGTTCCACCAGGTGAATGTCA | (SEQ ID NO: 38) |
| TaqMan primer: | (5′-3′): TTGGCAACAAGCTGAGCCGGG | (SEQ ID NO: 39) |

| GSTa3 mRNA (NM_000847) | | | | | | | |
|---|---|---|---|---|---|---|---|
| 1 | atacacatca | ggaggtggcc | ttgagaagct | gagcggagac | cggctagact | ttactcaaag | (SEQ ID NO: 40) |
| 61 | aaaccaagag | actgttacca | tggcagggaa | gcccaagctt | cactacttca | atggacgggg | |
| 121 | cagaatggag | cccatccggt | ggctcttggc | tgcagctgga | gtggagtttg | aagagaaatt | |
| 181 | tataggatct | gcagaagatt | tgggaaagtt | aagaaatgat | gggagtttga | tgttccagca | |
| 241 | agtaccaatg | gttgagattg | atgggatgaa | gttggtacag | accagagcca | ttctcaacta | |
| 301 | cattgccagc | aaatacaacc | tctacgggaa | agacataaag | gagagagccc | taattgatat | |
| 361 | gtatacagaa | ggtatggcag | atttgaatga | aatgatcctt | cttctgccct | tatgtcgacc | |
| 421 | tgaggaaaaa | gatgccaaga | ttgccttgat | caaagagaaa | acaaaaagtc | gctatttccc | |
| 481 | tgccttcgaa | aaagtgttac | agagccatgg | acaagactac | cttgttggca | acaagctgag | |
| 541 | ccgggctgac | attagcctgg | tggaacttct | ctactatgtg | gaagagcttg | actccagcct | |
| 601 | tatctccaac | ttccctctgc | tgaaggccct | gaaaaccaga | atcagcaacc | tgcccacggt | |
| 661 | gaagaagttt | ctacagcctg | gcagcccaag | gaagcctccc | gcagatgcaa | aagctttaga | |
| 721 | agaagccaga | aagattttca | ggttttaata | aagcagccat | ggaggctaag | aacatgcaag | |
| 781 | accaatattc | taaagttttg | caacaatgaa | gtgctttact | taagtgttga | ttgtgcctgt | |
| 841 | tgtaaagcta | atgaaccctt | tccaattata | tgctaattaa | ataataaaaa | ctcctatttg | |
| 901 | ctaacttaaa | aaaaaaaaaa | aaaaaaaaaa | aaaaaaa | | | |

From this sequence the forward primer, reverse primer and TaqMan primer can be identified using Primer Express® (Applied Biosystems). The primers shown below as SEQ ID NOS: 41-43 are preferred for the measurement of GSTa3, but other functional primers can be identified using SEQ ID NO: 40 and Primer Express® (Applied Biosystems) by one skilled in the art).

| | | |
|---|---|---|
| Forward primer: | (5′-3′): GCTGAAGGCCCTGAAAACC | (SEQ ID NO: 41) |
| Reverse primer: | (5′-3′): GGCTGCCAGGCTGTAGAAAC | (SEQ ID NO: 42) |
| TaqMan primer: | (5′-3′): AATCAGCAACCTGCCCACGGTGA | (SEQ ID NO: 43) |

| GSTa4 mRNA (NM_001512) | | | | | | | |
|---|---|---|---|---|---|---|---|
| 1 | agctcccgcg | cgctagagcc | gcctgctggt | ctcacccagc | cgggaccgct | gacctggcgc | (SEQ ID NO: 44) |
| 61 | tttgtgcggc | tccaggcctc | cgagtggact | ccagaaagcc | tgaaaagcta | tcatggcagc | |
| 121 | aaggcccaag | ctccactatc | ccaacggaag | aggccggatg | gagtccgtga | gatgggtttt | |
| 181 | agctgccgcc | ggagtcgagt | ttgatgaaga | atttctggaa | acaaaagaac | agttgtacaa | |
| 241 | gttgcaggat | ggtaaccacc | tgctgttcca | acaagtgccc | atggttgaaa | ttgacgggat | |
| 301 | gaagttggta | cagacccgaa | gcattctcca | ctacatagca | gacaagcaca | atctctttgg | |
| 361 | caagaacctc | aaggagagaa | ccctgattga | catgtacgtg | gaggggacac | tggatctgct | |
| 421 | ggaactgctt | atcatgcatc | ctttcttaaa | accagatgat | cagcaaaagg | aagtggttaa | |
| 481 | catggcccag | aaggctataa | ttagatactt | tcctgtgttt | gaaaagattt | taaggggtca | |
| 541 | cggacaaagc | tttcttgttg | gtaatcagct | gagccttgca | gatgtgattt | tactccaaac | |
| 601 | cattttagct | ctagaagaga | aaattcctaa | tatcctgtct | gcatttcctt | tcctccagga | |
| 661 | atacacagtg | aaactaagta | atatccctac | aattaagaga | ttccttgaac | ctggcagcaa | |
| 721 | gaagaagcct | ccccctgatg | aaatttatgt | gagaaccgtc | tacaacatct | ttaggccata | |
| 781 | aaacaacaca | tccatgtgtg | agtgacagtg | tgttcctaga | gatggtattg | tctacagtca | |
| 841 | tgtcttaatg | gatcccagct | ctgtcatggt | gctatctatg | tattaagttg | ggtcctaagt | |
| 901 | tgggtctttt | gtgtcaacga | gatcatctct | tctagaaata | tcaacctttt | ttgtccagta | |
| 961 | aataattgtt | aggggatctt | tattggaaaa | cttttttgga | gaggctggta | tttaagttag | |
| 1021 | atctgattgg | gctactcatg | tcctgtagcc | agttcatcct | cataataaga | atgggcagga | |
| 1081 | tctcttgttc | tctcctgagt | gtctttctac | tctcctgagc | gtctttctgc | tctccttatc | |
| 1141 | ctgttctctt | atccttatcc | cctccagtct | ctgcctaatt | tttagtgttt | aataacaacc | |
| 1201 | gaatgtctag | taaatgactc | tcctctgagc | tgtaataaat | aaaatggtag | taatgaatgc | |
| 1261 | aatcagtatt | agccaaaata | aagaatttat | gagtcattaa | aaaaaaaaaa | aaaaaaa | |

From this sequence the forward primer, reverse primer and TaqMan primer can be identified using Primer Express® (Applied Biosystems). The primers shown below as SEQ ID NOS: 45-47 are preferred for the measurement of GSTa4, but other functional primers can be identified using SEQ ID NO: 44 and Primer Express® (Applied Biosystems) by one skilled in the art).

| | | |
|---|---|---|
| Forward primer: | (5′-3′): GGTAACCACCTGCTGTTCCAA | (SEQ ID NO: 45) |
| Reverse primer: | (5′-3′): GCTTCGGGTCTGTACCAACTTC | (SEQ ID NO: 46) |
| TaqMan primer: | (5′-3′): AAGTGCCCATGGTTGAAATTGACGGG | (SEQ ID NO: 47) |

| GSTmu1 (variant 1) mRNA (NM_000561) | | | | | | | |
|---|---|---|---|---|---|---|---|
| 1 | ctctgagccc | tgctcggttt | aggcctgtct | gcggaatccg | caccaaccag | caccatgccc | (SEQ ID NO: 48) |
| 61 | atgatactgg | ggtactggga | catccgcggg | ctggcccacg | ccatccgcct | gctcctggaa | |
| 121 | tacacagact | caagctatga | ggaaaagaag | tacacgatgg | gggacgctcc | tgattatgac | |
| 181 | agaagccagt | ggctgaatga | aaaattcaag | ctgggcctgg | actttcccaa | tctgccctac | |
| 241 | ttgattgatg | gggctcacaa | gatcacccag | agcaacgcca | tcttgtgcta | cattgcccgc | |
| 301 | aagcacaacc | tgtgtgggga | gacagaagag | gagaagattc | gtgtggacat | tttggagaac | |
| 361 | cagaccatgg | acaaccatat | gcagctgggc | atgatctgct | acaatccaga | atttgagaaa | |
| 421 | ctgaagccaa | agtacttgga | ggaactccct | gaaaagctaa | agctctactc | agagtttctg | |
| 481 | gggaagcggc | catggtttgc | aggaaacaag | atcacttttg | tagattttct | cgtctatgat | |
| 541 | gtccttgacc | tccaccgtat | atttgagccc | aagtgcttgg | acgccttccc | aaatctgaag | |
| 601 | gacttcatct | cccgctttga | gggcttggag | aagatctctg | cctacatgaa | gtccagccgc | |
| 661 | ttcctcccaa | gacctgtgtt | ctcaaagatg | gctgtctggg | gcaacaagta | gggccttgaa | |
| 721 | ggccaggagg | tgggagtgag | gagcccatac | tcagcctgct | gcccaggctg | tgcagcgcag | |
| 781 | ctggactctg | catcccagca | cctgcctcct | cgttcctttc | tcctgtttat | tcccatcttt | |
| 841 | actcccaaga | cttcattgtc | cctcttcact | ccccctaaac | ccctgtccca | tgcaggccct | |
| 901 | ttgaagcctc | agctacccac | tatccttcgt | gaacatcccc | tcccatcatt | acccttccct | |
| 961 | gcactaaagc | cagcctgacc | ttccttcctg | ttagtggttg | tgtctgcttt | aaagggcctg | |
| 1021 | cctggcccct | cgcctgtgga | gctcagcccc | gagctgtccc | cgtgttgcat | gaaggagcag | |
| 1081 | cattgactgg | tttacaggcc | ctgctcctgc | agcatggtcc | ctgccttagg | cctacctgat | |
| 1141 | ggaagtaaag | cctcaaccac | a | | | | |

From this sequence the forward primer, reverse primer and TaqMan primer can be identified using Primer Express® (Applied Biosystems). The primers shown below as SEQ ID NOS: 49-51 are preferred for the measurement of GSTmu1, but other functional primers can be identified using SEQ ID NO: 48 and Primer Express® (Applied Biosystems) by one skilled in the art).

| | | |
|---|---|---|
| Forward primer: | (5′-3′): GCCCTTTGAAGCCTCAGCTA | (SEQ ID NO: 49) |
| Reverse primer: | (5′-3′): TTTAGTGCAGGGAAGGGTAATGA | (SEQ ID NO: 50) |
| TaqMan primer: | (5′-3′): CCACTATCCTTCGTGAACATCCCCTCC | (SEQ ID NO: 51) |

| GST mu 1 (variant 2) (NM_146421) | | |
|---|---|---|
| 1 | ctctgagccc tgctcggttt aggcctgtct gcggaatccg caccaaccag caccatgccc | (SEQ ID NO: 52) |
| 61 | atgatactgg ggtactggga catccgcggg ctggcccacg ccatccgcct gctcctggaa | |
| 121 | tacacagact caagctatga ggaaaagaag tacacgatgg gggacgctcc tgattatgac | |
| 181 | agaagccagt ggctgaatga aaaattcaag ctgggcctgg actttcccaa tctgccctac | |
| 241 | ttgattgatg gggctcacaa gatcacccag agcaacgcca tcttgtgcta cattgcccgc | |
| 301 | aagcacaacc tgtgtgggga gacagaagag gagaagattc gtgtggacat tttggagaac | |
| 361 | cagaccatgg acaaccatat gcagctgggc atgatctgct acaatccaga atttgagaaa | |
| 421 | ctgaagccaa agtacttgga ggaactccct gaaaagctaa agctctactc agagtttctg | |
| 481 | gggaagcggc catggtttgc aggaaacaag ggcttggaga agatctctgc ctacatgaag | |
| 541 | tccagccgct tcctcccaag acctgtgttc tcaaagatgg ctgtctgggg caacaagtag | |
| 601 | ggccttgaag gccaggaggt gggagtgagg agcccatact cagcctgctg cccaggctgt | |
| 661 | gcagcgcagc tggactctgc atcccagcac ctgcctcctc gttcctttct cctgtttatt | |
| 721 | cccatcttta ctcccaagac ttcattgtcc ctcttcactc cccctaaacc cctgtcccat | |
| 781 | gcaggccctt tgaagcctca gctacccact atccttcgtg aacatcccct cccatcatta | |
| 841 | cccttccctg cactaaagcc agcctgacct tccttcctgt tagtggttgt gtctgcttta | |
| 901 | aagggcctgc ctggcccctc gcctgtggag ctcagccccg agctgtcccc gtgttgcatg | |
| 961 | aaggagcagc attgactggt ttacaggccc tgctcctgca gcatggtccc tgccttaggc | |
| 1021 | ctacctgatg gaagtaaagc ctcaaccaca | |

From this sequence the forward primer, reverse primer and TaqMan primer can be identified using Primer Express® (Applied Biosystems). The primers shown below as SEQ ID NOS: 53-55 are preferred for the measurement of GSTmu1, but other functional primers can be identified using SEQ ID NO: 52 and Primer Express® (Applied Biosystems) by one skilled in the art).

| | | |
|---|---|---|
| Forward primer: | (5′-3′): TTTAGGCCTGTCTGCGGAAT | (SEQ ID NO: 53) |
| Reverse primer: | (5′-3′): GATGTCCCAGTACCCCAGTATCA | (SEQ ID NO: 54) |
| TaqMan primer: | (5′-3′): CGCACCAACCAGCACCATGCC | (SEQ ID NO: 55) |

| GST mu 3 mRNA (NM_000849) | | |
|---|---|---|
| 1 | ggttggttct gagaaggctt caaggaatag gcagacattt cagcaaggct gctgaggaag | (SEQ ID NO: 56) |
| 61 | gataggctgt ggaaattagg atgcagcact cctgcccggg tcccgcctcg gggtcgccag | |
| 121 | gccctgaacc ccaacgccgg cattagtcgc gcctgcgcac ggccctgtgg agccgcggag | |
| 181 | gcaagggacg gagaacgggg cggaggcgga gtcagggcgc ccgcgcgtgg gccccgcccc | |
| 241 | cttatgtcgg gtataaagcc cctcccgctc acagtttccc tagtcctcga aggctcggaa | |
| 301 | gcccgtcacc atgtcgtgcg agtcgtctat ggttctcggg tactgggata ttcgtgggct | |
| 361 | ggcgcacgcc atccgcctgc tcctggagtt cacggatacc tcttatgagg agaaacggta | |
| 421 | cacgtgcggg gaagctcctg actatgatcg aagccaatgg ctggatgtga aattcaagct | |
| 481 | agacctggac tttcctaatc tgccctacct cctggatggg aagaacaaga tcacccagag | |
| 541 | caatgccatc ttgcgctaca tcgctcgcaa gcacaacatg tgtggtgaga ctgaagaaga | |
| 601 | aaagattcga gtggacatca tagagaacca agtaatggat ttccgcacac aactgataag | |
| 661 | gctctgttac agctctgacc acgaaaaact gaagcctcag tacttggaag agctacctgg | |
| 721 | acaactgaaa caattctcca tgtttctggg gaaattctca tggtttgccg gggaaaagct | |
| 781 | cacctttgtg gattttctca cctatgatat cttggatcag aaccgtatat ttgaccccaa | |
| 841 | gtgcctggat gagttcccaa acctgaaggc tttcatgtgc cgttttgagg ctttggagaa | |
| 901 | aatcgctgcc tacttacagt ctgatcagtt ctgcaagatg cccatcaaca acaagatggc | |
| 961 | ccagtggggc aacaagcctg tatgctgagc aggaggcaga cttgcagagc ttgttttgtt | |
| 1021 | tcatcctgtc cgtaaggggt cagcgctctt gctttgctct tttcaatgaa tagcacttat | |
| 1081 | gttactggtg tccagctgag tttctcttgg gtataaaggc taaaagggaa aaaggatatg | |
| 1141 | tggagaatca tcaagatatg aattgaatcg ctgcgatact ggcatttccc tactccccaa | |
| 1201 | ctgagttcaa gggctgtagg ttcatgccca agccctgaga gtgggtacta gaaaaaacga | |
| 1261 | gattgcacag ttggagagag caggtgtgtt aaatgggact ggagtccctg tgaagactgg | |
| 1321 | gtgaggataa cacaagtaaa actgtggtac tgatggactt aaccggagtt cggaaaccgt | |
| 1381 | cctgtgtaca catgggagtt tagtgtgata aaggcagtat ttcagactgg tgggctagcc | |
| 1441 | aatagagttg ggacaattgc ttactcatta aaaataatag agccccactt gacactattc | |
| 1501 | actaaaatta atctggaatt taaggcccaa cattaaacac aaagctgttg aaataaaaaa | |
| 1561 | aaaaaaaaaa aa | |

From this sequence the forward primer, reverse primer and TaqMan primer can be identified using Primer Express® (Applied Biosystems). The primers shown below as SEQ ID NOS: 57-59 are preferred for the measurement of GSTmu3, but other functional primers can be identified using SEQ ID NO: 56 and Primer Express® (Applied Biosystems) by one skilled in the art).

| | | |
|---|---|---|
| Forward primer: | (5′-3′): CGCTGCGATACTGGCATTT | (SEQ ID NO: 57) |
| Reverse primer: | (5′-3′): GGGCTTGGGCATGAACCT | (SEQ ID NO: 58) |
| TaqMan primer: | (5′-3′): CCTACTCCCCAACTGAGTTCAAGGGCTG | (SEQ ID NO: 59) |

| GST mu 4 mRNA (NM_000850) | | |
|---|---|---|
| 1 | ggcgaggccg agcccctcct agtgcttccg gaccttgctc cctgaacact cggaggtggc | (SEQ ID NO: 60) |
| 61 | ggtggatctt actccttcca gccagtgagg atccagcaac ctgctccgtg cctcccgcgc | |
| 121 | ctgttggttg gaagtgacga ccttgaagat cggccggttg gaagtgacga ccttgaagat | |
| 181 | cggcgggcgc agcggggccg agggggcggg tctggcgcta ggtccagccc ctgcgtgccg | |
| 241 | ggaaccccag aggaggtcgc agttcagccc agctgaggcc tgtctgcaga atcgacacca | |
| 301 | accagcatca tgtccatgac actggggtac tgggacatcc gcgggctggc ccacgccatc | |
| 361 | cgcctgctcc tggaatacac agactcaagc tacgaggaaa agaagtatac gatgggggac | |
| 421 | gctcctgact atgacagaag ccagtggctg aatgaaaaat tcaagctggg cctggacttt | |
| 481 | cccaatctgc cctacttgat tgatggggct cacaagatca cccagagcaa cgccatcctg | |
| 541 | tgctacattg cccgcaagca caacctgtgt ggggagacag aagaggagaa gattcgtgtg | |
| 601 | gacattttgg agaaccaggc tatggacgtc tccaatcagc tggccagagt ctgctacagc | |
| 661 | cctgactttg agaaactgaa gccagaatac ttggaggaac ttcctacaat gatgcagcac | |
| 721 | ttctcacagt tcctggggaa gaggccatgg tttgttggag acaagatcac ctttgtagat | |
| 781 | ttcctcgcct atgatgtcct tgacctccac cgtatatttg agcccaactg cttggacgcc | |
| 841 | ttcccaaatc tgaaggactt catctcccgc tttgagggct tggagaagat ctctgcctac | |
| 901 | atgaagtcca gccgcttcct cccaaaacct ctgtacacaa gggtggctgt ctggggcaac | |
| 961 | aagtaatgcc ttgaaggcca ggaggtggga gtgaggagcc catactcagc ctgctgccca | |
| 1021 | ggctgtgcag cgcagctgga ctctgcatcc cagcacctgc ctcctcgttc ctttctcctg | |
| 1081 | tttattccca tctttacccc caagacttta ttgggcctct tcacttcccc taaacccctg | |
| 1141 | tcccatgcag gccctttgaa gcctcagcta cccactttcc ttcatgaaca tccccctccc | |
| 1201 | aacactaccc ttccctgcac taaagccagc ctgaccttcc ttcctgttag tggttgtatc | |
| 1261 | tgctttgaag ggcctacctg gcccctcgcc tgtggagctc agccctgagc tgtccccgtg | |
| 1321 | ttgcatgaca gcattgactg gtttacaggc cctgctcctg cagcatggcc cctgccttag | |
| 1381 | gcctacctga tcaaaataaa gcctcagcca caaaaaaaaa aaaaaaaaaa aaaaaa | |

From this sequence the forward primer, reverse primer and TaqMan primer can be identified using Primer Express® (Applied Biosystems). The primers shown below as SEQ ID NOS: 61-63 are preferred for the measurement of GSTmu4, but other functional primers can be identified using SEQ ID NO: 60 and Primer Express® (Applied Biosystems) by one skilled in the art).

| | | |
|---|---|---|
| Forward primer: | (5′-3′): GCAGCGCAGCTGGACTCT | (SEQ ID NO: 61) |
| Reverse primer: | (5′-3′): GGTAAAGATGGGAATAAACAGGAGAA | (SEQ ID NO: 62) |
| TaqMan primer: | (5′-3′): ATCCCAGCACCTGCCTCCTCGTTC | (SEQ ID NO: 63) |

| GST mu 5 mRNA (NM_000851) | | |
|---|---|---|
| 1 | tcctgggcct ctcaaagtct gagccccgct ccgctgatgc ctgtctgcag aatccgcacc | (SEQ ID NO: 64) |
| 61 | aaccagcacc atgcccatga ctctggggta ctgggacatc cgtgggctgg cccacgccat | |
| 121 | ccgcttgctc ctggaataca cagactcaag ctatgtggaa aagaagtaca cgctggggga | |
| 181 | cgctcctgac tatgacagaa gccagtggct gaatgaaaaa ttcaagctgg gcctggactt | |
| 241 | tcccaatctg ccctacttga ttgatggggc tcacaagatc acccagagca atgccatcct | |
| 301 | gcgctacatt gcccgcaagc acaacctgtg tggggagaca gaagaggaga agattcgtgt | |
| 361 | ggacattttg gagaaccagg ttatggataa ccacatggag ctggtcagac tgtgctatga | |
| 421 | cccagatttt gagaaactga agccaaaata cttggaggaa ctccctgaaa agctaaagct | |
| 481 | ctactcagag tttctgggga agcggccatg gtttgcagga gacaagatca cctttgtgga | |
| 541 | tttccttgcc tatgatgtcc ttgacatgaa gcgtatattt gagcccaagt gcttggacgc | |
| 601 | cttcctaaac ttgaaggact tcatctcccg ctttgagggt ttgaagaaga tctctgccta | |
| 661 | catgaagtcc agccaattcc tccgaggtct tttgtttgga aagtcagcta catggaacag | |
| 721 | caaatagggc ccagtgatgc cagaagatgg gagggaggag ccaaccttgc tgcctgcgac | |
| 781 | cctggaggac agcctgactc cctggacctg ccttcttcct ttttccttct ttctactctc | |
| 841 | ttctcttccc caaggcctca ttggcttcct ttcttctaac atcatccctc cccgcatcga | |
| 901 | ggctctttaa agcttcagct ccccactgtc ctccatcaaa gtccccctcc taacgtcttc | |
| 961 | ctttccctgc actaacgcca acctgactgc ttttcctgtc agtgcttttc tcttctttga | |
| 1021 | gaagccagac tgatctctga gctccctagc actgtcctca aagaccatct gtatgccctg | |
| 1081 | ctccctttgc tgggtcccta ccccagctcc gtgtgatgcc cagtaaagcc tgaaccatgc | |
| 1141 | ctgccatgtc ttgtcttatt ccctgaggct cccttgactc aggactgtgc tcgaattgtg | |
| 1201 | ggtggttttt tgtcttctgt tgtccacagc cagagcttag tggatgggtg tgtgtgtgtg | |
| 1261 | tgtgttgggg gtggtgatca ggcaggttca taaatttcct tggtcatttc tgccctctag | |
| 1321 | ccacatccct ctgttcctca ctgtggggat tactacagaa aggtgctctg tgccaagttc | |
| 1381 | ctcactcatt cgcgctcctg taggccgtct agaactggca tggttcaaag aggggctagg | |
| 1441 | ctgatgggga agggggctga gcagctccca ggcagactgc cttctttcac cctgtcctga | |
| 1501 | tagacttccc tgatctagat atccttcgtc atgacacttc tcaataaaac gtatcccacc | |
| 1561 | gtattgt | |

From this sequence the forward primer, reverse primer and TaqMan primer can be identified using Primer Express® (Applied Biosystems). The primers shown below as SEQ ID NOS: 65-67 are preferred for the measurement of GSTmu5, but other functional primers can be identified using SEQ ID NO: 64 and Primer Express® (Applied Biosystems) by one skilled in the art).

| | | |
|---|---|---|
| Forward primer: | (5′-3′): CAGCAAATAGGGCCCAGTGA | (SEQ ID NO: 65) |
| Reverse primer: | (5′-3′): GGGTCGCAGGCAGCAA | (SEQ ID NO: 66) |
| TaqMan primer: | (5′-3′): CCAGAAGATGGGAGGGAGGAGCCA | (SEQ ID NO: 67) |

| GST omega mRNA (NM_004832) | | |
|---|---|---|
| 1 | tgcgccacga tgtccgggga gtcagccagg agcttgggga agggaagcgc gcccccgggg | (SEQ ID NO: 68) |
| 61 | ccggtcccgg agggctcgat ccgcatctac agcatgaggt tctgcccgtt tgctgagagg | |
| 121 | acgcgtctag tcctgaaggc caagggaatc aggcatgaag tcatcaatat caacctgaaa | |
| 181 | aataagcctg agtggttctt taagaaaaat ccctttggtc tggtgccagt tctggaaaac | |
| 241 | agtcagggtc agctgatcta cgagtctgcc atcacctgtg agtacctgga tgaagcatac | |
| 301 | ccagggaaga agctgttgcc ggatgacccc tatgagaaag cttgccagaa gatgatctta | |
| 361 | gagttgtttt ctaaggtgcc atccttggta ggaagcttta ttagaagcca aaataaagaa | |
| 421 | gactatgctg gcctaaaaga agaatttcgt aaagaattta ccaagctaga ggaggttctg | |
| 481 | actaataaga agacgacctt ctttggtggc aattctatct ctatgattga ttacctcatc | |
| 541 | tggccctggt ttgaacggct ggaagcaatg aagttaaatg agtgtgtaga ccacactcca | |
| 601 | aaactgaaac tgtggatggc agccatgaag gaagatccca cagtctcagc cctgcttact | |
| 661 | agtgagaaag actggcaagg tttcctagag ctctacttac agaacagccc tgaggcctgt | |
| 721 | gactatgggc tctgaagggg gcaggagtca gcaataaagc tatgtctgat attttccttc | |
| 781 | agtaaaaaaa aaa | |

From this sequence the forward primer, reverse primer and TaqMan primer can be identified using Primer Express® (Applied Biosystems). The primers shown below as SEQ ID NOS: 69-71 are preferred for the measurement of GST omega, but other functional primers can be identified using SEQ ID NO: 68 and Primer Express® (Applied Biosystems) by one skilled in the art).

| | | |
|---|---|---|
| Forward primer: | (5′-3′): CCGCATCTACAGCATGAGGTT | (SEQ ID NO: 69) |
| Reverse primer: | (5′-3′): TCCCTTGGCCTTCAGGACTA | (SEQ ID NO: 70) |
| TaqMan primer: | (5′-3′): TGCCCGTTTGCTGAGAGGACGC | (SEQ ID NO: 71) |

The levels of GST mRNA may be quantitated using real time PCR system. More specifically, this protocol uses a 26 μl reaction composed of 1.0 μl DNA 10 ng/μl; 4.0 μl Forward MapPair primer; 4.0 μl 1 Reverse MapPair primer; and 71 μl of PCR mix solution: 25 μl PCR Buffer 10× with 15 mM MgCL₂ (Tris-CL₁, KC₁, (NH₄)2SO₄, 15 mM MgCL₂, pH 8.7 at 20C.); 1.25 μl 1 dNTP 2.5 mM; 2.6 μl Cresol Red 30%; 0.125 μl distilled H₂O and 0.045 μl Taq 5.0 U/ul.

Thermal Cycling Conditions:

1) Touchdown Thermocycle Protocol:

```
  1. 94° C. for 5 min
  2. 60° C. for 1 min
  3. 72° C. for 1 min
  4. 94° C. for 30 sec
  5. 58° C. for 1 min
  6. 72° C. for 1 min
  7. 94° C. for 30 sec
  8. 56° C. for 1 min
  9. 72° C. for 1 min
10. 94° C. for 30 sec
11. 54° C. for 1 min
12. 72° C. for 1 min
13. 94° C. for 30 sec
14. 52° C. for 1 min
15. 72° C. for 1 min
16. 94° C. for 1 min
17. 50° C. for 1 min
18. 72° C. for 1 min
19. 25 times to 16
20. hold at 8° C. for 24
```

Gel Electrophoresis:

Polyacrylamide gel electrophoresis will be performed on a Biorad DNA sequencer using 1×TBE (Tris-Borate-EDTA) buffer. All the samples will be run at 150V for 330 minutes. A 28 well-comb is used for lane formation, and 27 μl of each sample is loaded/lane. 7 ul of PCR marker (Promega) is loaded in the middle lane to identify the size of the PCR product. To visualize the PCR products, the gel will be stained in a solution of Ethidium Bromide for approximately 5 minutes. The stained gels will be photographed using the short wave UV on a ChemiImager 4000 Low Light Imaging System (Alpha Innotech Corporation). The image will be digitally-stored in TIFF format for data analysis as well as printed for hard copy using a thermal imagining printer.

Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

What is claimed is:

1. A method of determining the level of susceptibility of a subject to an environmental toxin capable of detoxification by a glutathione S-transferase comprising:
a. determining a first amount of one or more glutathione S-transferases present in a biological sample from the subject;
b. contacting the biological sample with the environmental toxin;
c. determining a second amount of one or more glutathione S-transferases present in the sample;
wherein the second amount of one or more glutathione S-transferases being lower than or similar to the first amount of one or more glutathione S-transferases indicates the subject having a higher level of susceptibility than a subject having the second amount of one or more glutathione S-transferases higher than the first amount of one or more glutathione S-transferases, and wherein the second amount of one or more glutathione S-transferases being higher than the first amount of one or more glutathione S-transferases indicates the subject having a lower level of susceptibility than a subject having a second amount of one or more glutathione S-transferases lower than or similar to the first amount of one or more glutathione S-transferases.

2. The method of claim 1 wherein the subject is a mammal.

3. The method of claim 1 wherein the subject is a human.

4. The method of claim 1 wherein the subject is a rodent.

5. The method of claim 1 wherein the subject is a mouse.

6. The method of claim 1 wherein the biological sample is selected from the group consisting of plasma, brain and urine.

7. The method of claim 1 wherein the one or more glutathione S-transferases is composed of subunits selected from the group consisting of: alpha, mu, pi and omega subunits of glutathione S-transferase.

8. The method of claim 1 wherein the one or more glutathione S-transferase is human glutathione S-transferase pi.

9. The method of claim 1 wherein the environmental toxin is a toxin wherein the toxin or a metabolite thereof interferes with Complex I respiration in a cell's electron transport chain.

10. The method of claim 1 wherein the environmental toxin is a toxin structurally similar to MPTP.

11. The method of claim 1 wherein the environmental toxin is selected from the group consisting of MPTP, MPPT, rotenone and paraquat.

12. The method of claim 1 wherein determining said first and second amounts of one or more glutathione S-transferases involve determining the amount of mRNA encoding said one or more glutathione S-transferases.

13. The method of claim 1 wherein determining said first and second amounts of one or more glutathione S-transferases involve the step of determining said one or more glutathione S-transferases by the method selected from the group consisting of: radioimmunoassay, enzyme immunoassay and immunoflurometric immuno assay.

14. The method of claim 12 wherein determining the amount of mRNA encoding said one or more glutathione S-transferases is determined using a primer selected from the group consisting of: SEQ ID NOS. 5-27, and SEQ ID NOS: 29-31, 33-35, 37-39, 40-43, 45-47, 49-51, 53-55, 57-59, 61-63, 65-67 and 69-71.

15. The method of claim 1 wherein determining said first and second amounts of one or more of one or more glutathione S-transferases involves determining the level of enzymatic activity corresponding to one or more glutathione S-transferases.

16. A method of determining risk of developing Parkinson's disease in a human comprising:
a. determining a first amount of one or more glutathione S-transferases present in a biological sample from the subject;
b. contacting the biological sample with the environmental toxin;
c. determining a second amount of one or more glutathione S-transferases present in the sample;
wherein the second amount of one or more glutathione S-transferases being lower than or similar to the first amount of one or more glutathione S-transferases indicates the subject having a higher level of risk of developing Parkinson's disease than a subject having the second amount of one or more glutathione S-transferases higher than the first amount of one or more glutathione S-transferases, and wherein the second amount of one or more glutathione S-transferases being higher than the first amount of one or more glutathione S-transferases indicates the subject having a lower level of risk of developing Parkinson's disease than a subject having a second amount of the one or more glutathione S-transferases lower than or similar to the first amount of the one or more glutathione S-transferases.

17. The method of claim 16 wherein the biological sample is selected from the group consisting of plasma, brain and urine.

18. The method of claim 16 wherein the one or more glutathione S-transferase is human glutathione S-transferase pi.

19. The method of claim 16 wherein the environmental toxin is a toxin or a metabolite thereof interferes with Complex I respiration in a cell's electron transport chain.

20. The method of claim 16 wherein the environmental toxin is a toxin structurally similar to MPTP.

21. The method of claim 16 wherein the environmental toxin is selected from the group consisting of MPTP, MPPX, plus rotenone and paraquat.

22. The method of claim 16 wherein determining said first and second amounts of the one or more glutathione S-transferases involve determining the amount of mRNA encoding said the one or more glutathione S-transferases.

23. The method of claim 16 wherein determining said first and second amounts of the one or more glutathione S-transferases involve the step of determining said glutathione S-transferase by the method selected from the group consisting of: radioimmunoassay, enzyme immunoassay and immunoflurometric immunoassay.

24. The method of claim 23 wherein determining the amount of mRNA encoding said at least one glutathione S-transferases is determined using a primer selected from the group consisting of: SEQ ID NOS: 29-31,33-35, 37-39,41-43, 45-47, 49-51, 53-55, 57-59, 61-63, 65-67, and 69-71.

25. The method of claim 16 wherein determining said first and second amounts of the one or more glutathione S-transferases involves determining the level of enzymatic activity corresponding to the one or more glutathione S-transferases.

26. A method to determine genetic susceptibility to an environmental toxin involving contacting a biological sample from a subject with a composition consisting essentially of one or more labeled probes each of which binds selectively to a region on mouse chromosome 1, from D1Mit113 to D1Mit293 under conditions in which one or more labeled probes form stable hybridization complexes with DNA in the region of mouse chromosome I from D1Mit113 to D1Mit293 and detecting the hybridization complex, wherein said hybridization complex is indicative of a genetic susceptibility to an environmental toxin.

27. A method to determine the susceptibility of a subject to develop Parkinson's disease, the method comprising: contacting a biological sample from a subject with a composition consisting essentially of a plurality of labeled probes each of which selectively binds to a region of human Glutathione S-transferase pi within a segment of human GSTP1 (chr11:69874218-69877056); GST mu (hGSTm4: chr1:110677671-110687021, hGSTm2: chr1:110689771-110696957, hGSTm3: chr1:110758175-110761973, hGSTm1:chr1:110709530-110715415; or GST alpha (hGSTa1:chr6:52658645-52670705, hGSTa2:chr6:52617245-52630341; hGSTa3:chr6:52763514-52776547, hGSTa4:chr6:52844814-52862163 under conditions which a plurality of labeled probes form stable hybridization complexes with a segment of human GSTP1 (chr11:69874218-69877056); GST mu (hGSTm4: chr1:10677671-110687021, hGSTm2: chr1:110689771-110696957, hGSTm3: chr1:110758175-110761973, hGSTm1:chr1:110709530-110715415; or GST alpha (hGSTa1:chr6:52658645-52670705, hGSTa2:chr6:52617245-52630341; hGSTa3:chr6: 52763514-52776547, hGSTa4:chr6: 52844814-52862163; and detecting the hybridization complex wherein said hybridization complex is indicative of increased susceptibility of a subject to develop Parkinson's disease.


SEQUENCE LISTING (71 sequences)

SEQ ID NO: 1 - DNA, 630 bp, Mus sp.
atgccgccgtacaccattgtctacttcccaagtcgagggcggtgtgaggccatgcgcatgctgctgactgaccagggccagagctggaaggaggaggtggttaccatagatacctggatgcaaggcttgctcaagcccacttgtctgtatgggcagcttcctaagtttgaggatggagacctcaccctttaccaatctaatgccatcttgagacaccttggccgctctttggggctttatgggaaaaaccagagggaggccgcccaggtggatatggtgaatgatggggtagaggaccttcgcggcaaatatggcaccatgatctacagaaactatgagaatggtaagaatgactacgtgaaggccctgcctgggcatctgaagccttttgagaccctgctgtcccagaaccagggaggcaaagctttcatcgtgggtgaccagatctcctttgccgattacaacttgctggacctgctgctgatccaccaagtcctggcccctggctgcctggacaacttccccctgctctctgcctatgtggctcgcctcagtgcccggcccaagatcaaggcctttctgtcctccccggaacatgtgaaccgtcccatcaatggcaatggcaaacag

SEQ ID NO: 2 - DNA, 350 bp, Mus sp.
ggccatgcgcatgctgctgactgaccagggccagagctggaaggaggaggtggtgggcagcttcctaagtttgaggatggagacctcaccctttaccaatgactacgtgaaggccctgcctgggcatctgaagccttttgagaccctgctgtcccagaaccagggaggcaacagatctcctttgccgattacaacttgctggacctgctgctgatccaccaagtcctggcccctggctgcctggacaacttccccctgctctctgcctatgtggctcgcctcagtgcccggcccaagatcaaggcctttctgtcctccccggaacatgtgaaccgtcccatcaatggcaa

SEQ ID NO: 3 - DNA, 350 bp, Homo sapiens
ggccctgcgcatgctgctggcagatcagggccagagctggaaggaggaggtggtgggcagctccccaagttccaggacggagacctcaccctgtaccaatgactatgtgaaggcactgcccgggcaactgaagccttttgagaccctgctgtcccagaaccagggaggcaacagatctccttcgctgactacaacctgctggacttgctgctgatccatgaggtcctagcccctggctgcctggatgcgttccccctgctctcagcatatgtggggcgcctcagtgcccggcccaagctcaaggccttcctggcctcccctgagtacgtgaacctccccatcaatggcaa

SEQ ID NO: 4 - DNA, 630 bp, Mus sp.
atgccaccatacaccattgtctacttcccagttcgagggcggtgtgaggccatgcgaatgctgctggctgaccagggccagagctggaaggaggaggtggttaccatagatacctggatgcaaggcttgctcaagcccacttgtctgtatgggcagctccccaagtttgaggatggagacctcaccctttaccaatctaatgccatcttgagacaccttggccgctctttggggctttatgggaaaaaccagagggaggccgcccagatggatatggtgaatgatggggtggaggaccttcgcggcaaatatgtcaccctcatctacaccaactatgagaatggtaagaatgactacgtgaaggccctgcctgggcatctgaagccttttgagaccctgctgtcccagaaccagggaggcaaagctttcatcgtgggtgaccagatctcctttgccgattacaacttgctggacctgctgctgatccaccaagtcctggcccctggctgcctggacaacttccccctgctctctgcctatgtggctcgcctcagtgcccggcccaagatcaaggcctttctgtcctccccggaacatgtgaaccgtcccatcaatggcaatggcaaacag

SEQ ID NO: 5 - DNA, 26 bp, Artificial sequence
  Not a naturally occurring sequence
gcacttgccagcttcaggtgaattca

SEQ ID NO: 6 - DNA, 26 bp, Artificial sequence
  Not a naturally occurring sequence
gtcaggtagcttcaggcagaaccaca

SEQ ID NO: 7 - DNA, 15 bp, Artificial sequence
  Not a naturally occurring sequence
cacctgggccgctcc

SEQ ID NO: 8 - DNA, 19 bp, Artificial sequence
  Not a naturally occurring sequence
tgtccaccagtgctgcctc

SEQ ID NO: 9 - DNA, 25 bp, Artificial sequence
  Not a naturally occurring sequence
tcgggctctatggcaaagaccaaca

SEQ ID NO: 10 - DNA, 16 bp, Artificial sequence
  Not a naturally occurring sequence
tgtgaggccatgcgca

SEQ ID NO: 11 - DNA, 21 bp, Artificial sequence
  Not a naturally occurring sequence
tggtaaccacctcctccttcc

SEQ ID NO: 12 - DNA, 24 bp, Artificial sequence
  Not a naturally occurring sequence
ctgctgactgaccagggccagagc

SEQ ID NO: 13 - DNA, 18 bp, Artificial sequence
  Not a naturally occurring sequence
caggctgagcagggctga

SEQ ID NO: 14 - DNA, 17 bp, Artificial sequence
  Not a naturally occurring sequence
gcccgggtccagctctt

SEQ ID NO: 15 - DNA, 30 bp, Artificial sequence
  Not a naturally occurring sequence
attgccctggttgaactcctctaccatgtg

SEQ ID NO: 16 - DNA, 16 bp, Artificial sequence
  Not a naturally occurring sequence
gcaatggccgggaagc

SEQ ID NO: 17 - DNA, 21 bp, Artificial sequence
  Not a naturally occurring sequence
acctgatgcactccattctgc

SEQ ID NO: 18 - DNA, 25 bp, Artificial sequence
  Not a naturally occurring sequence
cgtgcttcactacttcaatgcccgg

SEQ ID NO: 19 - DNA, 22 bp, Artificial sequence
  Not a naturally occurring sequence
caggcaccttatattcgaaccc

SEQ ID NO: 20 - DNA, 18 bp, Artificial sequence
  Not a naturally occurring sequence
cgcaggccacaaagtcct

SEQ ID NO: 21 - DNA, 26 bp, Artificial sequence
  Not a naturally occurring sequence
cgtgcctggatgccttcctaaacctg

SEQ ID NO: 22 - DNA, 18 bp, Artificial sequence
  Not a naturally occurring sequence
atgccatcctgcgctacc

SEQ ID NO: 23 - DNA, 21 bp, Artificial sequence
  Not a naturally occurring sequence
tcctttcctcctctgtctccc

SEQ ID NO: 24 - DNA, 21 bp, Artificial sequence
  Not a naturally occurring sequence
tggccgcaagcacaacctgtg

SEQ ID NO: 25 - DNA, 21 bp, Artificial sequence
  Not a naturally occurring sequence
caccgaatatttgagcccaag

SEQ ID NO: 26 - DNA, 20 bp, Artificial sequence
  Not a naturally occurring sequence
caaagcgacccatgaagtcc

SEQ ID NO: 27 - DNA, 23 bp, Artificial sequence
  Not a naturally occurring sequence
cctggacgccttcccaaacctga

SEQ ID NO: 28 - DNA, 737 bp, Homo sapiens
ggagtttcgccgccgcagtcttcgccaccatgccgccctacaccgtggtctatttcccagttcgaggccgctgcgcggccctgcgcatgctgctggcagatcagggccagagctggaaggaggaggtggtgaccgtggagacgtggcaggagggctcactcaaagcctcctgcctatacgggcagctccccaagttccaggacggagacctcaccctgtaccagtccaataccatcctgcgtcacctgggccgcacccttgggctctatgggaaggaccagcaggaggcagccctggtggacatggtgaatgacggcgtggaggacctccgctgcaaatacatctccctcatctacaccaactatgaggcgggcaaggatgactatgtgaaggcactgcccgggcaactgaagccttttgagaccctgctgtcccagaaccagggaggcaagaccttcattgtgggagaccagatctccttcgctgactacaacctgctggacttgctgctgatccatgaggtcctagcccctggctgcctggatgcgttccccctgctctcagcatatgtggggcgcctcagcgcccggcccaagctcaaggccttcctggcctcccctgagtacgtgaacctccccatcaatggcaacgggaaacagtgagggttggggggactctgagcgggaggcagagtttgccttcctttctccaggaccaataaaatttctaagagagct

SEQ ID NO: 29 - DNA, 24 bp, Artificial sequence
  Not a naturally occurring sequence
cctgtaccagtccaataccatcct

SEQ ID NO: 30 - DNA, 20 bp, Artificial sequence
  Not a naturally occurring sequence
cctgctggtccttcccatag

SEQ ID NO: 31 - DNA, 19 bp, Artificial sequence
  Not a naturally occurring sequence
cgtcacctgggccgcaccc

SEQ ID NO: 32 - DNA, 833 bp, Homo sapiens
aaatacattaggacctggaattcagttgtcgagccaggacggtgacagcgtttaacaaagcttagagaaacctccaggagactgctatcatggcagagaagcccaagctccactacttcaatgcacggggcagaatggagtccacccggtggctcctggctgcagctggagtagagtttgaagagaaatttataaaatctgcagaagatttggacaagttaagaaatgatggatatttgatgttccagcaagtgccaatggttgagattgatgggatgaagctggtgcagaccagagccattctcaactacattgccagcaaatacaacctctatgggaaagacataaaggagagagccctgattgatatgtatatagaaggtatagcagatttgggtgaaatgatcctccttctgcccgtatgtccacctgaggaaaaagatgccaagcttgccttgatcaaagagaaaataaaaaatcgctacttccctgcctttgaaaaagtcttaaagagccatggacaagactaccttgttggcaacaagctgagccgggctgacattcatctggtggaacttctctactacgtcgaggagcttgactccagtcttatctccagcttccctctgctgaaggccctgaaaaccagaatcagcaacctgcccacagtgaagaagtttctacagcctggcagcccaaggaagcctcccatggatgagaaatctttagaagaagcaaggaagattttcaggttttaataacgcagtcatggaggccaagaacttgcaataccaatgttctaaagttttgcaacaataaagtactttacctaa

SEQ ID NO: 33 - DNA, 24 bp, Artificial sequence
  Not a naturally occurring sequence
agaaacctccaggagactgctatc

SEQ ID NO: 34 - DNA, 17 bp, Artificial sequence
  Not a naturally occurring sequence
tgccccgtgcattgaag

SEQ ID NO: 35 - DNA, 24 bp, Artificial sequence
  Not a naturally occurring sequence
tggcagagaagcccaagctccact

SEQ ID NO: 36 - DNA, 976 bp, Homo sapiens
tattgagaggaacaaagagcttataaatacattaggacctggaattcggttgtccagccacaaaggtgacagcatttaacaaagcttagagaaacctccaggagactgctaccatggcagagaagcccaagctccactactccaatatacggggcagaatggagtccatccggtggctcctggctgcagctggagtagagtttgaagagaaatttataaaatctgcagaagatttggacaagttaagaaatgatggatatttgatgttccagcaagtgccaatggttgagattgatgggatgaagctggtgcagaccagagccattctcaactacattgccagcaaatacaacctctatgggaaagacataaaggagaaagccctgattgatatgtatatagaaggtatagcagatttgggtgaaatgatccttcttctgccctttactcaacctgaggaacaagatgccaagcttgccttgatccaagagaaaacaaaaaatcgctacttccctgcctttgaaaaagtcttaaagagccacggacaagactaccttgttggcaacaagctgagccgggctgacattcacctggtggaacttctctactacgtggaagagcttgactctagccttatttccagcttccctctgctgaaggccctgaaaaccagaatcagtaacctgcccacagtgaagaagtttctacagcctggcagcccaaggaagcctcccatggatgagaaatctttagaagaatcaaggaagattttcaggttttaataaaccagccatagaggtcaagaacatgcaagaccagtattctaaagttttgcaacaattaagtgctttacctaagtgttgattgtgcctgttgtgaagctaatgaactctttcaaattatatgctaattaaataatacaactcctattcacccacttagttaaaattgatttcttctcattaggatctgatgt

SEQ ID NO: 37 - DNA, 22 bp, Artificial sequence
  Not a naturally occurring sequence
gagccacggacaagactacctt

SEQ ID NO: 38 - DNA, 22 bp, Artificial sequence
  Not a naturally occurring sequence
aagttccaccaggtgaatgtca

SEQ ID NO: 39 - DNA, 21 bp, Artificial sequence
  Not a naturally occurring sequence
ttggcaacaagctgagccggg

SEQ ID NO: 40 - DNA, 937 bp, Homo sapiens
atacacatcaggaggtggccttgagaagctgagcggagaccggctagactttactcaaagaaaccaagagactgttaccatggcagggaagcccaagcttcactacttcaatggacggggcagaatggagcccatccggtggctcttggctgcagctggagtggagtttgaagagaaatttataggatctgcagaagatttgggaaagttaagaaatgatgggagtttgatgttccagcaagtaccaatggttgagattgatgggatgaagttggtacagaccagagccattctcaactacattgccagcaaatacaacctctacgggaaagacataaaggagagagccctaattgatatgtatacagaaggtatggcagatttgaatgaaatgatccttcttctgcccttatgtcgacctgaggaaaaagatgccaagattgccttgatcaaagagaaaacaaaaagtcgctatttccctgccttcgaaaaagtgttacagagccatggacaagactaccttgttggcaacaagctgagccgggctgacattagcctggtggaacttctctactatgtggaagagcttgactccagccttatctccaacttccctctgctgaaggccctgaaaaccagaatcagcaacctgcccacggtgaagaagtttctacagcctggcagcccaaggaagcctcccgcagatgcaaaagctttagaagaagccagaaagattttcaggttttaataaagcagccatggaggctaagaacatgcaagaccaatattctaaagttttgcaacaatgaagtgctttacttaagtgttgattgtgcctgttgtaaagctaatgaaccctttccaattatatgctaattaaataataaaaactcctatttgctaacttaaaaaaaaaaaaaaaaaaaaaaaaaaaaaa

SEQ ID NO: 41 - DNA, 19 bp, Artificial sequence
  Not a naturally occurring sequence
gctgaaggccctgaaaacc

SEQ ID NO: 42 - DNA, 20 bp, Artificial sequence
  Not a naturally occurring sequence
ggctgccaggctgtagaaac

SEQ ID NO: 43 - DNA, 23 bp, Artificial sequence
  Not a naturally occurring sequence
aatcagcaacctgcccacggtga

SEQ ID NO: 44 - DNA, 1317 bp, Homo sapiens
agctcccgcgcgctagagccgcctgctggtctcacccagccgggaccgctgacctggcgctttgtgcggctccaggcctccgagtggactccagaaagcctgaaaagctatcatggcagcaaggcccaagctccactatcccaacggaagaggccggatggagtccgtgagatgggttttagctgccgccggagtcgagtttgatgaagaatttctggaaacaaaagaacagttgtacaagttgcaggatggtaaccacctgctgttccaacaagtgcccatggttgaaattgacgggatgaagttggtacagacccgaagcattctccactacatagcagacaagcacaatctctttggcaagaacctcaaggagagaaccctgattgacatgtacgtggaggggacactggatctgctggaactgcttatcatgcatcctttcttaaaaccagatgatcagcaaaaggaagtggttaacatggcccagaaggctataattagatactttcctgtgtttgaaaagattttaaggggtcacggacaaagctttcttgttggtaatcagctgagccttgcagatgtgattttactccaaaccattttagctctagaagagaaaattcctaatatcctgtctgcatttcctttcctccaggaatacacagtgaaactaagtaatatccctacaattaagagattccttgaacctggcagcaagaagaagcctccccctgatgaaatttatgtgagaaccgtctacaacatctttaggccataaaacaacacatccatgtgtgagtgacagtgtgttcctagagatggtattgtctacagtcatgtcttaatggatcccagctctgtcatggtgctatctatgtattaagttgggtcctaagttgggtcttttgtgtcaacgagatcatctcttctagaaatatcaaccttttttgtccagtaaataattgttaggggatctttattggaaaacttttttggagaggctggtatttaagttagatctgattgggctactcatgtcctgtagccagttcatcctcataataagaatgggcaggatctcttgttctctcctgagtgtctttctactctcctgagcgtctttctgctctccttatcctgttctcttatccttatcccctccagtctctgcctaatttttagtgtttaataacaaccgaatgtctagtaaatgactctcctctgagctgtaataaataaaatggtagtaatgaatgcaatcagtattagccaaaataaagaatttatgagtcattaaaaaaaaaaaaaaaaaaa

SEQ ID NO: 45 - DNA, 21 bp, Artificial sequence
  Not a naturally occurring sequence
ggtaaccacctgctgttccaa

SEQ ID NO: 46 - DNA, 22 bp, Artificial sequence
  Not a naturally occurring sequence
gcttcgggtctgtaccaacttc

SEQ ID NO: 47 - DNA, 26 bp, Artificial sequence
  Not a naturally occurring sequence
aagtgcccatggttgaaattgacggg

SEQ ID NO: 48 - DNA, 1161 bp, Homo sapiens
ctctgagccctgctcggtttaggcctgtctgcggaatccgcaccaaccagcaccatgcccatgatactggggtactgggacatccgcgggctggcccacgccatccgcctgctcctggaatacacagactcaagctatgaggaaaagaagtacacgatgggggacgctcctgattatgacagaagccagtggctgaatgaaaaattcaagctgggcctggactttcccaatctgccctacttgattgatggggctcacaagatcacccagagcaacgccatcttgtgctacattgcccgcaagcacaacctgtgtggggagacagaagaggagaagattcgtgtggacattttggagaaccagaccatggacaaccatatgcagctgggcatgatctgctacaatccagaatttgagaaactgaagccaaagtacttggaggaactccctgaaaagctaaagctctactcagagtttctggggaagcggccatggtttgcaggaaacaagatcacttttgtagattttctcgtctatgatgtccttgacctccaccgtatatttgagcccaagtgcttggacgccttcccaaatctgaaggacttcatctcccgctttgagggcttggagaagatctctgcctacatgaagtccagccgcttcctcccaagacctgtgttctcaaagatggctgtctggggcaacaagtagggccttgaaggccaggaggtgggagtgaggagcccatactcagcctgctgcccaggctgtgcagcgcagctggactctgcatcccagcacctgcctcctcgttcctttctcctgtttattcccatctttactcccaagacttcattgtccctcttcactccccctaaacccctgtcccatgcaggccctttgaagcctcagctacccactatccttcgtgaacatcccctcccatcattacccttccctgcactaaagccagcctgaccttccttcctgttagtggttgtgtctgctttaaagggcctgcctggcccctcgcctgtggagctcagccccgagctgtccccgtgttgcatgaaggagcagcattgactggtttacaggccctgctcctgcagcatggtccctgccttaggcctacctgatggaagtaaagcctcaaccaca

SEQ ID NO: 49 - DNA, 20 bp, Artificial sequence
  Not a naturally occurring sequence
gccctttgaagcctcagcta

SEQ ID NO: 50 - DNA, 23 bp, Artificial sequence
  Not a naturally occurring sequence
tttagtgcagggaagggtaatga

SEQ ID NO: 51 - DNA, 27 bp, Artificial sequence
  Not a naturally occurring sequence
ccactatccttcgtgaacatcccctcc

SEQ ID NO: 52 - DNA, 1050 bp, Homo sapiens
ctctgagccctgctcggtttaggcctgtctgcggaatccgcaccaaccagcaccatgcccatgatactggggtactgggacatccgcgggctggcccacgccatccgcctgctcctggaatacacagactcaagctatgaggaaaagaagtacacgatgggggacgctcctgattatgacagaagccagtggctgaatgaaaaattcaagctgggcctggactttcccaatctgccctacttgattgatggggctcacaagatcacccagagcaacgccatcttgtgctacattgcccgcaagcacaacctgtgtggggagacagaagaggagaagattcgtgtggacattttggagaaccagaccatggacaaccatatgcagctgggcatgatctgctacaatccagaatttgagaaactgaagccaaagtacttggaggaactccctgaaaagctaaagctctactcagagtttctggggaagcggccatggtttgcaggaaacaagggcttggagaagatctctgcctacatgaagtccagccgcttcctcccaagacctgtgttctcaaagatggctgtctggggcaacaagtagggccttgaaggccaggaggtgggagtgaggagcccatactcagcctgctgcccaggctgtgcagcgcagctggactctgcatcccagcacctgcctcctcgttcctttctcctgtttattcccatctttactcccaagacttcattgtccctcttcactccccctaaacccctgtcccatgcaggccctttgaagcctcagctacccactatccttcgtgaacatcccctcccatcattacccttccctgcactaaagccagcctgaccttccttcctgttagtggttgtgtctgctttaaagggcctgcctggcccctcgcctgtggagctcagccccgagctgtccccgtgttgcatgaaggagcagcattgactggtttacaggccctgctcctgcagcatggtccctgccttaggcctacctgatggaagtaaagcctcaaccaca

SEQ ID NO: 53 - DNA, 20 bp, Artificial sequence
  Not a naturally occurring sequence
tttaggcctgtctgcggaat

SEQ ID NO: 54 - DNA, 23 bp, Artificial sequence
  Not a naturally occurring sequence
gatgtcccagtaccccagtatca

SEQ ID NO: 55 - DNA, 21 bp, Artificial sequence
  Not a naturally occurring sequence
cgcaccaaccagcaccatgcc

SEQ ID NO: 56 - DNA, 1572 bp, Homo sapiens
ggttggttctgagaaggcttcaaggaataggcagacatttcagcaaggctgctgaggaaggataggctgtggaaattaggatgcagcactcctgcccgggtcccgcctcggggtcgccaggccctgaaccccaacgccggcattagtcgcgcctgcgcacggccctgtggagccgcggaggcaagggacggagaacggggcggaggcggagtcagggcgcccgcgcgtgggccccgcccccttatgtcgggtataaagcccctcccgctcacagtttccctagtcctcgaaggctcggaagcccgtcaccatgtcgtgcgagtcgtctatggttctcgggtactgggatattcgtgggctggcgcacgccatccgcctgctcctggagttcacggatacctcttatgaggagaaacggtacacgtgcggggaagctcctgactatgatcgaagccaatggctggatgtgaaattcaagctagacctggactttcctaatctgccctacctcctggatgggaagaacaagatcacccagagcaatgccatcttgcgctacatcgctcgcaagcacaacatgtgtggtgagactgaagaagaaaagattcgagtggacatcatagagaaccaagtaatggatttccgcacacaactgataaggctctgttacagctctgaccacgaaaaactgaagcctcagtacttggaagagctacctggacaactgaaacaattctccatgtttctggggaaattctcatggtttgccggggaaaagctcacctttgtggattttctcacctatgatatcttggatcagaaccgtatatttgaccccaagtgcctggatgagttcccaaacctgaaggctttcatgtgccgttttgaggctttggagaaaatcgctgcctacttacagtctgatcagttctgcaagatgcccatcaacaacaagatggcccagtggggcaacaagcctgtatgctgagcaggaggcagacttgcagagcttgttttgtttcatcctgtccgtaaggggtcagcgctcttgctttgctcttttcaatgaatagcacttatgttactggtgtccagctgagtttctcttgggtataaaggctaaaagggaaaaaggatatgtggagaatcatcaagatatgaattgaatcgctgcgatactggcatttccctactccccaactgagttcaagggctgtaggttcatgcccaagccctgagagtgggtactagaaaaaacgagattgcacagttggagagagcaggtgtgttaaatgggactggagtccctgtgaagactgggtgaggataacacaagtaaaactgtggtactgatggacttaaccggagttcggaaaccgtcctgtgtacacatgggagtttagtgtgataaaggcagtatttcagactggtgggctagccaatagagttgggacaattgcttactcattaaaaataatagagccccacttgacactattcactaaaattaatctggaatttaaggcccaacattaaacacaaagctgttgaaataaaaaaaaaaaaaaaaaa

SEQ ID NO: 57 - DNA, 19 bp, Artificial sequence
  Not a naturally occurring sequence
cgctgcgatactggcattt

SEQ ID NO: 58 - DNA, 18 bp, Artificial sequence
  Not a naturally occurring sequence
gggcttgggcatgaacct

SEQ ID NO: 59 - DNA, 28 bp, Artificial sequence
  Not a naturally occurring sequence
cctactccccaactgagttcaagggctg

SEQ ID NO: 60 - DNA, 1436 bp, Homo sapiens
ggcgaggccgagcccctcctagtgcttccggaccttgctccctgaacactcggaggtggcggtggatcttactccttccagccagtgaggatccagcaacctgctccgtgcctcccgcgcctgttggttggaagtgacgaccttgaagatcggccggttggaagtgacgaccttgaagatcggcgggcgcagcggggccgagggggcgggtctggcgctaggtccagcccctgcgtgccgggaaccccagaggaggtcgcagttcagcccagctgaggcctgtctgcagaatcgacaccaaccagcatcatgtccatgacactggggtactgggacatccgcgggctggcccacgccatccgcctgctcctggaatacacagactcaagctacgaggaaaagaagtatacgatgggggacgctcctgactatgacagaagccagtggctgaatgaaaaattcaagctgggcctggactttcccaatctgccctacttgattgatggggctcacaagatcacccagagcaacgccatcctgtgctacattgcccgcaagcacaacctgtgtggggagacagaagaggagaagattcgtgtggacattttggagaaccaggctatggacgtctccaatcagctggccagagtctgctacagccctgactttgagaaactgaagccagaatacttggaggaacttcctacaatgatgcagcacttctcacagttcctggggaagaggccatggtttgttggagacaagatcacctttgtagatttcctcgcctatgatgtccttgacctccaccgtatatttgagcccaactgcttggacgccttcccaaatctgaaggacttcatctcccgctttgagggcttggagaagatctctgcctacatgaagtccagccgcttcctcccaaaacctctgtacacaagggtggctgtctggggcaacaagtaatgccttgaaggccaggaggtgggagtgaggagcccatactcagcctgctgcccaggctgtgcagcgcagctggactctgcatcccagcacctgcctcctcgttcctttctcctgtttattcccatctttacccccaagactttattgggcctcttcacttcccctaaacccctgtcccatgcaggccctttgaagcctcagctacccactttccttcatgaacatccccctcccaacactacccttccctgcactaaagccagcctgaccttccttcctgttagtggttgtatctgctttgaagggcctacctggcccctcgcctgtggagctcagccctgagctgtccccgtgttgcatgacagcattgactggtttacaggccctgctcctgcagcatggcccctgccttaggcctacctgatcaaaataaagcctcagccacaaaaaaaaaaaaaaaaaaaaaaaaa

SEQ ID NO: 61 - DNA, 18 bp, Artificial sequence
  Not a naturally occurring sequence
gcagcgcagctggactct

SEQ ID NO: 62 - DNA, 26 bp, Artificial sequence
  Not a naturally occurring sequence
ggtaaagatgggaataaacaggagaa

SEQ ID NO: 63 - DNA, 24 bp, Artificial sequence
  Not a naturally occurring sequence
atcccagcacctgcctcctcgttc

SEQ ID NO: 64 - DNA, 1567 bp, Homo sapiens
tcctgggcctctcaaagtctgagccccgctccgctgatgcctgtctgcagaatccgcaccaaccagcaccatgcccatgactctggggtactgggacatccgtgggctggcccacgccatccgcttgctcctggaatacacagactcaagctatgtggaaaagaagtacacgctgggggacgctcctgactatgacagaagccagtggctgaatgaaaaattcaagctgggcctggactttcccaatctgccctacttgattgatggggctcacaagatcacccagagcaatgccatcctgcgctacattgcccgcaagcacaacctgtgtggggagacagaagaggagaagattcgtgtggacattttggagaaccaggttatggataaccacatggagctggtcagactgtgctatgacccagattttgagaaactgaagccaaaatacttggaggaactccctgaaaagctaaagctctactcagagtttctggggaagcggccatggtttgcaggagacaagatcacctttgtggatttccttgcctatgatgtccttgacatgaagcgtatatttgagcccaagtgcttggacgccttcctaaacttgaaggacttcatctcccgctttgagggtttgaagaagatctctgcctacatgaagtccagccaattcctccgaggtcttttgtttggaaagtcagctacatggaacagcaaatagggcccagtgatgccagaagatgggagggaggagccaaccttgctgcctgcgaccctggaggacagcctgactccctggacctgccttcttcctttttccttctttctactctcttctcttccccaaggcctcattggcttcctttcttctaacatcatccctccccgcatcgaggctctttaaagcttcagctccccactgtcctccatcaaagtccccctcctaacgtcttcctttccctgcactaacgccaacctgactgcttttcctgtcagtgcttttctcttctttgagaagccagactgatctctgagctccctagcactgtcctcaaagaccatctgtatgccctgctccctttgctgggtccctaccccagctccgtgtgatgcccagtaaagcctgaaccatgcctgccatgtcttgtcttattccctgaggctcccttgactcaggactgtgctcgaattgtgggtggttttttgtcttctgttgtccacagccagagcttagtggatgggtgtgtgtgtgtgtgtgttgggggtggtgatcaggcaggttcataaatttccttggtcatttctgccctctagccacatccctctgttcctcactgtggggattactacagaaaggtgctctgtgccaagttcctcactcattcgcgctcctgtaggccgtctagaactggcatggttcaaagaggggctaggctgatggggaagggggctgagcagctcccaggcagactgccttctttcaccctgtcctgatagacttccctgatctagatatccttcgtcatgacacttctcaataaaacgtatcccaccgtattgt

SEQ ID NO: 65 - DNA, 20 bp, Artificial sequence
  Not a naturally occurring sequence
cagcaaatagggcccagtga

SEQ ID NO: 66 - DNA, 16 bp, Artificial sequence
  Not a naturally occurring sequence
gggtcgcaggcagcaa

SEQ ID NO: 67 - DNA, 24 bp, Artificial sequence
  Not a naturally occurring sequence
ccagaagatgggagggaggagcca

SEQ ID NO: 68 - DNA, 793 bp, Homarus gammarus
tgcgccacgatgtccggggagtcagccaggagcttggggaagggaagcgcgcccccggggccggtcccggagggctcgatccgcatctacagcatgaggttctgcccgtttgctgagaggacgcgtctagtcctgaaggccaagggaatcaggcatgaagtcatcaatatcaacctgaaaaataagcctgagtggttctttaagaaaaatccctttggtctggtgccagttctggaaaacagtcagggtcagctgatctacgagtctgccatcacctgtgagtacctggatgaagcatacccagggaagaagctgttgccggatgacccctatgagaaagcttgccagaagatgatcttagagttgttttctaaggtgccatccttggtaggaagctttattagaagccaaaataaagaagactatgctggcctaaaagaagaatttcgtaaagaatttaccaagctagaggaggttctgactaataagaagacgaccttctttggtggcaattctatctctatgattgattacctcatctggccctggtttgaacggctggaagcaatgaagttaaatgagtgtgtagaccacactccaaaactgaaactgtggatggcagccatgaaggaagatcccacagtctcagccctgcttactagtgagaaagactggcaaggtttcctagagctctacttacagaacagccctgaggcctgtgactatgggctctgaagggggcaggagtcagcaataaagctatgtctgatattttccttcagtaaaaaaaaaa

SEQ ID NO: 69 - DNA, 21 bp, Artificial sequence
  Not a naturally occurring sequence
ccgcatctacagcatgaggtt

SEQ ID NO: 70 - DNA, 20 bp, Artificial sequence
  Not a naturally occurring sequence
tcccttggccttcaggacta

SEQ ID NO: 71 - DNA, 22 bp, Artificial sequence
  Not a naturally occurring sequence
tgcccgtttgctgagaggacgc